(19) United States
(12) Patent Application Publication
(10) Pub. No.: US 2024/0226166 A1
(43) Pub. Date: Jul. 11, 2024

(54) CD8(+) Stem-Like Chronic Memory Cell Based Therapies and Compositions Related Thereto

(71) Applicant: Emory University, Atlanta, GA (US)

(72) Inventors: Daniel Yunmin Chang, Atlanta, GA (US); Rafi Ahmed, Atlanta, GA (US); Tahseen H. Nasti, Snellville, GA (US)

(21) Appl. No.: 18/577,171

(22) PCT Filed: Jul. 6, 2022
(86) PCT No.: PCT/US2022/036205

§ 371 Date: Jan. 5, 2024

Related U.S. Application Data
(60) Provisional application No. 63/218,741, filed on Jul. 6, 2021.

(51) Int. Cl. A61K 35/17; A61K 39/00; A61K 45/06; C07K 14/705; C07K 16/28; C12N 5/0783

(52) U.S. Cl. CPC A61K 35/17; A61K 39/4611; A61K 39/4631; A61K 39/464411; A61K 45/06; C07K 14/70517; C07K 14/70564; C07K 16/2818; C07K 16/2827; C12N 5/0636; A61K 2039/505

(57) ABSTRACT

This disclosure relates to CD8 positive stem-like chronic memory cells for uses in managing diseases and conditions associated with T cell exhaustion and compositions related thereto. In certain embodiments. the CD8 positive cells are PD-1 positive or PD-1 negative. CD62L positive. CD127 positive, and CD44 positive. In certain embodiments. this disclosure relates to methods of treating cancer. chronic viral infections. or chronic diseases comprising administering to a patient in need thereof an effective amount of CD8 positive stem-like chronic memory cells optionally in combination with checkpoint inhibitors. In certain embodiments. the CD8 positive stem-like chronic memory cells are derived from the patient to be treated. are optionally expanded ex vivo, and optionally express a chimeric antigen receptor.

### CROSS-REFERENCE TO RELATED APPLICATIONS

This application claims the benefit of U.S. Provisional Application No. 63/218,741 filed Jul. 6, 2021. The entirety of this application is hereby incorporated by reference for all purposes.

### STATEMENT REGARDING FEDERALLY SPONSORED RESEARCH OR DEVELOPMENT

This invention was made with government support under AI030048 awarded by the National Institutes of Health. The government has certain rights in the invention.

### BACKGROUND

CD8 positive T cells in the thymus migrate to the spleen and lymphoid organs. These T cells interact with antigen presenting cells (APCs) as a step in the process of removing foreign agents or undesirable cells, e.g., cancerous cells. T cell exhaustion refers to a state were these antigen specific CD8 T cells are dysfunctional or physical eliminated typically observed after long term exposure to a viral infection or cancers. Exhausted T cells are characterized by increased expression of co-inhibitory receptors such as PD-1 (Programmed cell death protein 1) and CTLA-4. These receptors and their ligands are often referred to as checkpoint molecules. Blocking these receptor ligand interactions, e.g., with anti-PD-1 antibodies and CTLA-4 antibodies, are therapeutic strategies clinically approved for the treatment of certain cancers. Unfortunately, these therapeutics are not universally effective. Thus, there is a need to identify improvements.

Jansen et al. report that tumor-infiltrating T cells are comprised of two functionally distinct subsets: a TCF1+stem-like CD8 T cell population, and their progeny, a clonally related terminally differentiated population that express high levels of checkpoint molecules. Nature, 2019, 576, 465-470.

Siddiqui et al. report intratumoral TCF1⁺, PD-1⁺, and CD8⁺ T cells with stem-like properties promote tumor control in response to vaccination and checkpoint blockade immunotherapy. Immunity, 2019, 50(1):195-211.

Im et al. report PD-1⁺ stemlike CD8 T cells are resident in lymphoid tissues during persistent LCMV infection. PNAS, 2020, 117 (8) 4292-4299.

Gong et al. report tumor-infiltrating CD62L⁺PD-1⁻CD8 T cells retain proliferative potential via Bcl6 expression and replenish effector T cells within the tumor. PLOS ONE, 2020, 15(8): e0237646.

References cited herein are not an admission of prior art.

### SUMMARY

This disclosure relates to CD8 positive stem-like chronic memory cells for uses in managing diseases and conditions associated with T cell exhaustion and compositions related thereto. In certain embodiments, the CD8 positive cells are PD-1 positive or PD-1 negative, CD62L (L-selectin) positive, CD127 positive (Interleukin 7 receptor alpha chain), and CD44 positive. In certain embodiments, this disclosure relates to methods of treating cancer, chronic viral infections, or chronic diseases comprising administering to a patient in need thereof an effective amount of CD8 positive stem-like chronic memory cells optionally in combination with checkpoint inhibitors. In certain embodiments, the CD8 positive stem-like chronic memory cells are derived from the patient to be treated, are optionally expanded ex vivo, and optionally express a chimeric antigen receptor.

In certain embodiments, this disclosure relates to methods of isolating CD8 positive stem-like chronic memory cells comprising: obtaining a sample from a subject, purifying cells in the sample that are PD-1 positive and CD8 positive providing PD1 and CD8 positive cells; purifying cells from the PD-1 and CD8 positive cells that are CD62L positive, providing CD62L, CD8, and CD62L positive cells.

In certain embodiments, this disclosure relates to compositions of CD8 positive stem-like chronic memory cells made by the process of purifying cells from a sample that are PD-1 positive and CD8 positive providing PD-1 and CD8 positive cells; purifying cells from the PD1 and CD8 positive cells that are CD62L positive providing PD-1, CD8, CD62L, CD127, and CD44 positive cells.

In certain embodiments, this disclosure relates to compositions of CD8 positive stem-like chronic memory cells made by the process of purifying cells from a sample that are PD-1 positive and CD8 positive providing PD1 and CD8 positive cells; purifying cells from the PD1 and CD8 positive cells that are CD62L positive providing PD-1, CD8, CD62L, and CD127 positive cells.

In certain embodiments, this disclosure relates to compositions of CD8 positive stem-like chronic memory cells are PD-1 positive CD127 positive, CD62L positive CCR7 positive (C-C Motif Chemokine Receptor 7), TIM3 negative (T-cell immunoglobulin and mucin domain 3), TOX positive (Thymocyte selection-associated high mobility group box protein), and normal or elivated TCF1 (T cell factor 1) expression.

In certain embodiments, this disclosure relates to methods of treating cancer comprising administering to a patient in need thereof an effective amount of CD8 positive stem-like chronic memory cells wherein the PD-1 and CD8 positive stem-like chronic memory cells are replicated ex vivo prior to administration optionally in combination with another chemotherapy or radiation treatment.

In certain embodiments, the CD8 positive stem-like chronic memory cells or replicated cells thereof are derived from the patient (autologous) or derived from a person other than the patient. In certain embodiments, the PD-1 and CD8 positive stem-like chronic memory cells or replicated cells thereof are derived from a person other than the patient who recovered from a cancer therapy.

In certain embodiments, the CD8 positive stem-like chronic memory cells comprise a recombinant vector encoding a chimeric antigen receptor and express the chimeric antigen receptor providing cells that targets specific antigens, e.g., cancer antigens or viral antigens.

In certain embodiments, the CD8 positive stem-like chronic memory cells are administered in combination a checkpoint inhibitor. In certain embodiments, the checkpoint inhibitor is an anti-PD1 antibody, anti-PD-L1 antibody, anti-CTLA4 antibody, or combinations thereof. In certain embodiments, the checkpoint inhibitor is an anti-PDI antibody or anti-PD-L1 antibody selected from pembrolizumab, nivolumab, cemiplimab, atezolizumab, dostarlimab, durvalumab, and avelumab. In certain embodiments, the anti-CTLA4 antibody is ipilimumab or tremelimumab.

In certain embodiments, the cancer is a hematological cancer, myeloma, leukemia, lymphoma, basal cell carcinoma, bladder cancer, breast cancer, cervical cancer, colorectal cancer, endometrial cancer, esophageal carcinoma, gastric cancer, head and neck cancer, hepatocellular carcinoma, Hodgkin's lymphoma, malignant pleural mesothelioma, melanoma, Merkel cell carcinoma, lung cancer, small cell lung cancer, non-small cell cancer, lymphoma, renal cell carcinoma, solid tumors, squamous cell carcinoma, stomach cancer, or urothelial carcinoma.

In certain embodiments, the checkpoint inhibitor is a combination of nivolumab with ipilimumab useful for the treatment of renal cell carcinoma, colorectal cancer, hepatocellular carcinoma, non-small cell lung cancer (NSCLC), or malignant pleural mesothelioma.

In certain embodiments, this disclosure relates to methods of treating chronic viral infection comprising administering to a subject in need thereof an effective amount of CD8 positive stem-like chronic memory cells. In certain embodiments, the chronic viral infection is selected from HBV, HCV, and HIV. In certain embodiments, the composition of cells is administered in combination with another antiviral agent.

In certain embodiments, the CD8 positive stem-like chronic memory cells are CD62L positive and CD127 positive. In certain embodiments, the PD-1 and CD8 positive stem-like chronic memory cells are replicated ex vivo prior to administration. In certain embodiments, the CD8 positive stem-like chronic memory cells or replicated cells thereof are derived from the patient or derived from a person other than the patient. In certain embodiments, the CD8 positive stem-like chronic memory cells or replicated cells thereof are derived from a person other than the patient who recovered from an anti-viral therapy.

In certain embodiments, this disclosure relates to methods of treating chronic disease comprising administering to a subject in need thereof an effective amount of CD8 positive stem-like chronic memory cells.

In certain embodiments, this disclosure relates to methods of isolating CD8 positive stem-like chronic resource cells comprising, obtaining a sample from a subject, purifying cells in the sample that are PD-1 positive and CD8 positive providing PD1 and CD8 positive cells; purifying cells from the PD-1 and CD8 positive cells providing cells that express TCF1, are CD44 positive, and have no or low expression of Tim3, CD39 negative, or combination of these markers or other markers as disclosed herein, providing isolated CD8 positive stem-like chronic resource cells.

In certain embodiments, the method further comprises the step of expanding the isolated CD8 positive stem-like chronic resource cells.

In certain embodiments, cells as reported herein are isolated from blood, tumors, lymph nodes, or metastases. In certain embodiments, isolation is by using flow cytometer optionally in combination combination with the use of gene markers as disclosed herein (e.g., TCF1).

In certain embodiments, cells as reported herein are plated in a well(s) with media with cytokines and rested without or in the absence of beads or agonists that stimulate the T cell receptor (TCR).

In certain embodiments, an aliquot of the cells are taken pre-isolation or post-isolation for flow cytometry, PCR, western blot confirmation of expression of gene markers disclosed herein, (e.g., TCF1).

In certain embodiments, the method further comprises the step of resting the isolated CD8 positive stem-like chronic resource cells for a sufficient time that expression of CD127 and CD62L is detected.

In certain embodiments, expression of CD127 and CD62L is detected by flow cytometery.

In certain embodiments, resting is in vitro, e.g., in a cell growth medium, or in vivo.

In certain embodiments, a resting period is for a sufficient time until the cells are ready to be utilize for therapy and/or for expression of chimeric antigen receptors, e.g., when the expression of CD127 and CD62L are observed, e.g., as detected by flow cytometry.

In certain embodiments, resting is in the absence of T cell receptor agonists.

In certain embodiments, the T cell receptor agonist is a cognate peptide, antigen-presenting cell, antibody or small molecule agonist of CD3 and/or T cell receptor.

In certain embodiments, the subject is the same subject from which the PD1 and CD8 positive cells were originally obtained, or the subject is not the same subject from which the the PD1 and CD8 positive cells were originally obtained.

In certain embodiments, the CD8 positive stem-like chronic resource cells are engineered to express a chimeric antigen receptor. In certain embodiments, the CD8 positive stem-like chronic resource cells are administered or infused into a subject for use in a medical therapy. In certain embodiments, the medical therapy is the treatment of cancer, chronic viral infections, or chronic diseases. In certain embodiments, the CD8 positive stem-like chronic resource cells are administered or infused to a subject in combination with a checkpoint inhibitor or combinations thereof.

In certain embodiments, this disclosure relates to composition made by the processes provided described herein. In certain embodiments, this disclosure relates to kits or articles of manufacture, comprising cells or compositions made by the processes provided described herein as disclosed herein and instructions for use by, e.g., a healthcare professional. The kits or articles of manufacture may include a vial or a syringe containing the formulation as described herein.

### BRIEF DESCRIPTION OF THE SEVERAL VIEWS OF THE DRAWING(S)

FIGS. 1A-E shows data indicating antigen-specific CD8 T cells with expression of canonical memory markers emerge one year after the clearance of chronic LCMV infection.

FIG. 1A shows data on serum viral kinetics in Armstrong, Cl-13, and GK C1-13 LCMV models.

FIG. 1B shows data on tetramer staining of CD8 T cells at one-year post-infection.

FIG. 1C shows data on median fluorescence intensity (MFI) of PD-1 splenic antigen-specific CD8 T cells.

FIG. 1D shows data on expression and frequency of CD127 in splenic antigen-specific CD8 T cells.

FIG. 1E shows data on the expression and frequency of CD62L in splenic antigen-specific CD8 T cells.

FIGS. 2A-2D show data indicating PD-1+CD127+CD44+CD62L+ chronic memory CD8 T cells are transcriptionally distinct from acute memory cells and have resemblance to stem-like cells during chronic infection.

FIG. 2A shows a sorting strategy of persistent antigen-specific cells in acute and chronic infection models using CD62L expression.

FIG. 2B shows principal components analysis of transcriptional data.

FIG. 2C shows normalized expression counts of various molecules by subsets.

FIG. 2D shows data from Gene Set Enrichment Analysis (GSEA) of CD62L+ and CD62L− subsets identified after the clearance of chronic LCMV infection compared to gene signatures of CXCR5+Tim3− and CXCR5−Tim3+ subsets during chronic LCMV infection.

FIG. 3 shows data indicating chronic memory CD62L+ adaptive transfer leads to the highest reduction in the chronic viral burden. Serum viral titers were assessed 14 days after adaptive transfer of different memory subsets. The group of mice receiving the transfer of chronic memory CD62L+ cells showed the best control of viral infection which could have implications on therapeutic effects of these cells in the context of chronic viral infection, hematologic cancer, and solid tumors.

FIG. 4 illustrates an experiment to show stem-like resource CD8 T cells upregulate cannonical memory markers after antigen withdrawal. CD4-depleted mice were infected with chronic LCMV. Stem-like resource (PD-1+CD44+Tim3−CD73+CD39−) and terminally-differentiated (PD-1+CD44+Tim3+CD73−CD39+) CD8 T cells were sorted and 250,000 cells were transferred into congenically-marked LCMV-immune recipient mice.

FIG. 5A illustrates experiments indicating CD62L+ subsets have superior recall response to acute LCMV rechallenge. Mice were infected with acute or chronic LCMV. Splenic antigen-specific CD8 T cells were sorted based on CD62L expression>1 year post-infection and equal numbers of each subset was transferred into congenically-marked naïve recipient. One day later, recipients were challenged with acute LCMV.

FIG. 5B shows kinetics of donor CD8 T cells in the PBMC.

FIG. 5C shows data from tetramer staining and numbers of donors in the spleen 40 days after acute LCMV infection.

FIG. 5D showed data from Ki67 staining and quantification of homeostatic proliferation (TCF1+Ki67+) of donor CD8 T cells in the spleen.

FIG. 5E shows data on TOX expression/median fluorescence intensity (MFI) in the donor CD8 T cells 40 days after acute LCMV infection.

FIG. 5F shows data on PD-1 expression/median fluorescence intensity (MFI) in the donor CD8 T cells 40 days after acute LCMV infection.

FIG. 5G shows data on intracellular cytokine staining and quantification following GP33 and GP276 peptide stimulation.

FIGS. 6A-6G show data indicating stem-like resource CD8 T cells persist and acquire memory phenotype after antigen withdrawal.

FIG. 6A illustrates a method to obtain stem-like resource CD8 T cells (PD-1+CD44+Tim3−CD39−CD73+) and terminally-differentiated cells (PD-1+CD44+Tim3+CD39+CD73−) sorted from congenically mice chronically infected with LCMV. These subsets of cells were then transferred to LCMV-immune mice. Fate and phenotype were assessed 30-days after cessation of antigen stimulation.

FIG. 6B shows data on the number of CD45.2+ donor cells in the spleen.

FIG. 6C shows data on the normalized number that are GP33+CD45.2+.

FIG. 6D shows data on the normalized number that are GP276+CD45.2+.

FIG. 6E shows data on phenotypic characterization of GP33+GP276+ donor stem-like resource and terminally-differentiated CD8 T cells 30-days after antigen withdrawal.

FIG. 6F shows data on the frequency of CD127+ and CD62L+ donor CD8 T cells 30-days after antigen withdrawal.

FIG. 6G illustrates a method where stem-like and terminally-differentiated cells were sorted as described in FIG. 6A and transcriptional analyses were performed in GP33+GP276+ donor cells 60-days after antigen withdrawal.

FIGS. 7A-H show data indicating chronic CD62L+ memory cells provide superior proliferative burst and persist contributing to viral control after rechallenge with chronic viral infection.

FIG. 7A illustrates a method where mice were infected with acute or chronic LCMV. Splenic antigen-specific CD8 T cells were sorted based on CD62L expression greater than 1 year post-infection and equal numbers of each subset was transferred into congenically-marked naïve recipient. One day later, recipients were challenged with chronic LCMV.

FIG. 7B shows data on longitudinal analysis of the frequency of donors in PBMC after rechallenge.

FIG. 7C shows data on longitudinal analysis of the number of donors in spleen after rechallenge.

FIG. 7D shows data on MFI of TOX in antigen-specific donor and endogenous CD8 T cells at day 7 post-rechallenge.

FIG. 7E shows data on phenotype and number of donor stem-like resource CD8 T cells at day 14 post-rechallenge.

FIG. 7F shows data on the number of donor CD8 T cells in the bone marrow, lung, liver, kidney, and brain at d14 post-rechallenge.

FIG. 7G shows data on viral titers in the serum day 21 post-rechallenge.

FIG. 7H shows data on viral titers in the bone marrow day 14 post-rechallenge.

FIG. 8A-8C show data on adoptive therapy using chronic stem-like memory CD8 T cells provide superior tumor regression compared to acute memory.

FIG. 8A illustrates a method where mice were infected with acute or chronic LCMV. Splenic antigen-specific CD8 T cells were sorted based on CD62L expression>1 year post-infection and equal numbers of each subset was transferred into congenically-marked recipient mice bearing bilateral flank subcutaneous B16 melanoma tumors expressing the LCMV GP at d10 post-implantation.

FIG. 8B shows longitudinal analysis data of the tumor growth in each group.

FIG. 8C shows summary data of tumor growth kinetics.

### DETAILED DISCUSSION

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein. It is also to be understood that the terminology used herein is for the purpose of describing particular embodiments or example only and is not intended to be limiting. In describing and claiming the present disclosure, the following terminology will be used.

The articles “a” and “an” are used herein to refer to one or to more than one (i.e., to at least one) of the grammatical object of the article. By way of example, “an element” means one element or more than one element.

As used herein, the term “autologous” is meant to refer to any material derived from the same individual to which it is later to be re-introduced into the individual.

“Allogeneic” refers to any material derived from a different animal of the same species.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

One can positively isolate T cells from lymphy tissues, whole blood, buffy coat, mononuclear cells or bone marrow by using conventional cell sorting techniques such as florescent activated cells sorting. One can also use antibodies that bind T cells markers bound to magnetic material. One can remove the beads using a magnet and use an agent to release the T cells from the beads. One can also isolate purified T cells by immunomagnetic negative selection. Non-T cells can be targeted for removal with antibodies conjugated to magnetic material recognizing specific surface markers. Unwanted cells are labelled with antibodies and may be separated using a magnet.

This disclosure contemplates methods disclosed herein, wherein T cells are obtained by positive or negative selection of T cells with T cell markers or non T cell markers. This disclosure contemplates methods disclosed herein, wherein the T cell markers are CD3, CD4, CD8, or combinations thereof.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “expand” as used herein refers to increasing the number of T cells through replication in a growth medium. In certain embodiments, the T cells are expanded ex vivo to increase T cells in number relative to the number of cells originally isolated. In another embodiment, the T cells that are expanded ex vivo increase in number relative to other cell types in a culture. The term “ex vivo,” as used herein, refers to cells that have been removed from a living organism, (e.g., a human) and propagated outside the organism (e.g., in a culture dish, test tube, or bioreactor).

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide/disaccharide/polysaccharide, medium chain fatty acids, and/or optionally nucleotides. Typical components in a growth medium include amino acids (histidine, isoleucine, leucine, lysine, methionine, phenylalanine, threonine, tryptophan, valine and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth medium may contain phenol red as a pH indication. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate.

Various growth mediums are known in the art. Minimal Essential Medium (MEM) is a term of art referring to a growth medium that contains calcium chloride, potassium chloride, magnesium sulfate, sodium chloride, sodium phosphate and sodium bicarbonate, essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin M), choline, and inositol (originally known as vitamin B8). Dulbecco's modified Eagle's medium (DMEM) is a growth medium which contains additional components such as glycine, serine, and ferric nitrate with increased amounts of vitamins, amino acids, and glucose.

“Isolated” means altered or removed from the natural state. For example, a nucleic acid or a peptide or a cell naturally present in a living animal is not “isolated,” but the same nucleic acid or peptide or cell partially or completely separated from the coexisting materials of its natural state is “isolated.” An isolated nucleic acid or protein can exist in substantially purified form, or can exist in a non-native environment such as, for example, a host cell. In another non-limiting example, a T cell removed from a subject is “isolated”.

By the term “modified” or “modifying” as used herein, is meant a changed state or structure of a molecule or cell of the disclosure. Cells may be modified through the introduction of nucleic acids. For example, a T cell can be modified to contain a chimeric antigen receptor (CAR). The cells may be modified to contain vector that encodes the CAR and expresses the CAR which incorporates into the cell membrane.

A “vector” is a composition of matter which comprises a recombinant nucleic acid which can be used to deliver the nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. The term should also be construed to include non-plasmid and non-viral compounds which facilitate transfer of nucleic acid into cells, such as, for example, polylysine compounds, liposomes, and the like.

As used herein “endogenous” refers to any material from or produced inside an organism, cell, tissue, or system.

As used herein, the term “exogenous” refers to any material introduced from or produced outside an organism, cell, tissue, or system.

To “treat” a disease, as the term is used herein, means to reduce the frequency or severity of at least one sign or symptom of a disease or disorder experienced by a subject. It is not intended to be limited to the situation in which the disease or disorder is entirely eradicated. In certain embodiments, administering a composition for treatment with cells may be by parenteral administration or implantation. “Parenteral” administration of a composition includes, e.g., subcutaneous (s.c.), intravenous (i.v.), intramuscular (i.m.), or intrasternal injection, or infusion techniques.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control. It may also be identified by a change in relevant biomarker or gene expression profile, such as PSA for prostate cancer, HER2 for breast cancer, or others.

The cancer to be treated in the context of the present disclosure may be any type of cancer or tumor. These tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may be derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

Also contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genito-urinary apparatus and, more particularly, childhood acute lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the oesophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, lymphoproliferative disorders, macroglobulinemia, malignant mesothelioma, malignant thymoma, medulloblastoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-melanoma skin cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, rectal cancer, renal cell cancer, cancer of the renal pelvis and ureter, retinoblastoma, rhabdomyosarcoma, cancer of the salivary glands, sarcoidosis, sarcomas, skin cancer, small intestine cancer, soft tissue sarcoma, squamous neck cancer, stomach cancer, pineal and supratentorial primitive neuroectodermal tumors, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

A “chemotherapy agent,” “chemotherapeutic,” “anti-cancer agent,” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art. When “an immunologically effective amount,” “an autoimmune disease-inhibiting effective amount,” or “therapeutic amount” is indicated, the precise amount of the compositions of the present disclosure to be administered can be determined by a physician or researcher with consideration of individual differences in age, weight, tumor size, extent of infection or metastasis, and condition of the patient (subject).

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline, and murine mammals. Preferably, the subject is a human patient.

In certain embodiments, this disclosure relates to methods of treating cancer, chronic viral infections, or chronic diseases comprising administering to a patient in need thereof an effective amount of CD8 positive stem-like chronic memory cells comprising a CAR optionally in combination with checkpoint inhibitors. In certain embodiments, the CD8 positive stem-like chronic memory cells are derived from the patient to be treated, are optionally expanded ex vivo, and optionally express a chimeric antigen receptor.

As used herein, a “chimeric antigen receptor” or “CAR” refers to a protein receptor, which introduces an antigen specificity, via an antigen binding domain, onto cells (immune cells) to which it is expressed (for example cells disclosed herein) thus combining the antigen binding properties of the antigen binding domain with the cell activity. A CAR typically includes an extracellular antigen-binding domain (ectodomain), a transmembrane domain and an intracellular signaling domain. The intracellular signaling domain generally contains at least one immunoreceptor tyrosine-based activation motif (ITAM) signaling domain, e.g., derived from CD3zeta, and optionally at least one costimulatory signaling domain, e.g. derived from CD28 or 4-1BB.

In order to improve the ability of immune cells to kill cancerous cells, cells can be isolated from the blood of a patient in a mannor as disclosed herein and genetically altered to express chimeric antigen receptors (CARs) to specifically target proteins expressed on the surface of cancerous cells and stimulate an immune response. When put back into the patient, the cells attack the cancerous cells. Brentjens et al. report that T cells altered to bind CD19 can induce remissions of cancer in adults with chemotherapy-refractory acute lymphoblastic leukemia. Sci Transl Med, 2013, 5(177): 177ra38.

In certain embodiments, the chimeric antigen receptor specifically binds (EGFR) epidermal growth factor receptor, (HER2) human epidermal growth factor receptor 2, (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3[Fuca1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen. In certain embodiments, the chimeric antigen receptor is an antibody single-chain variable fragment (scFv).

Whole blood is composed of plasma, red blood cells (RBCs; or erythrocytes), platelets, and nucleated white blood cells, also referred to as leukocytes. The leukocytes can be further categorized into mononuclear cells and polymorphonuclear cells (or granulocytes). There are different techniques to obtain peripheral blood mononuclear cells (PBMCs), polymorphonuclear cells, leukocytes, or specific cell subsets, e.g., isolate specific cells directly by using flow cytometry, depleting red blood cells, centrifugation, and/or apheresis.

In a typical procedure, T cells and other immune cells are purified and isolated from blood or bone marrow. For example, T cells are collected via apheresis, a process that withdraws blood from the body and removes one or more blood components (such as plasma, platelets, or other white blood cells). The remaining blood is then returned back into the body. The cells are exposed to a recombinant vector, such as a lentiviral vector, that infects the cells in a way that a chimeric antigen receptor (CAR) protein is produced and presented in the cell membrane.

Before and/or after infecting the isolated cells with the recombinant vector, the cells may be induced to replicate. The genetically modified cells may be expanded by growing cells in the laboratory until there are sufficient number of them. Optionally, these CAR cells are frozen. The modified cells are then administered back to the patient.

In certain embodiments, the targeting sequence in a chimeric antigen receptor refers to any variety of polypeptide sequences capable of selectively binding to a targeted associated molecule. The targeting sequences may be derived from variable binding regions of antibodies, single chain antibodies, and antibody mimetics. In certain embodiments, targeting sequence is a single-chain variable fragment (scFv) derived from an antibody. The targeting sequence is typically connected to intracellular domains by a hinge/transmembrane region, commonly derived from CD8 or IgG4. The intracellular domains may contain co-stimulatory domains such as CD80, CD86, 4-1BBL, OX40L and CD70 and/or CD28 linked to the cytoplasmic signaling domain of CD3zeta. See Sadelain et al. The basic principles of chimeric antigen receptor (CAR) design, Cancer Discov. 2013, 3(4): 388-398.

Peripheral blood mononuclear cells (PBMCs) may be isolated by leukapheresis. T cells can be enriched by mononuclear cells counter-flow elutriation and expanded by addition of anti-CD3/CD28 antibody coated paramagnetic beads for activation of T cells. Cells may be expanded, harvested, and cryopreserved in infusible medium sometime after the subject has had an allogeneic stem-cell transplantation.

Cells may be obtained by isolation from peripheral blood and optionally purified by fluorescent activated cells sorting e.g., mixing cells with fluorescent antibodies or other fluorescent agents (molecular beacons) and separating the cells by flow cytometry based fluorescent sorting. Another option for cells sorting is to provide magnetic particles that are conjugated to specific binding agents, such as antibodies against a particular antigen on a target cells surface. After mixing with a sample, the antibody bound cells are put through a purification column containing a matrix composed of ferromagnetic spheres. When placed on a magnetic separator, the spheres amplify the magnetic field. The unlabeled cells pass through while the magnetically labeled cells are retained within the column. The flow-through can be collected as the unlabeled cells fraction. After a short washing step, the column is removed from the separator, and the magnetically labeled cells are eluted from the column.

CD3 is expressed on T cells as it is associated with the T cells receptor (TCR). The majority of TCR are made up of alpha beta chains (alpha beta T-cells). Alpha beta T-cells typically become double-positive intermediates (CD4+CD8+) which mature into single-positive (CD4+CD8−) T helper cells or (CD4−CD8+) cytotoxic T cells. T helper cells interact with antigen presenting dendritic cells and B cells. Upon activation with cognate antigen by dendritic cells, antigen specific CD4 T cells can differentiate to become various types of effector CD4 T cells with specific roles in promoting immune responses.

Immune cells may be isolated and separated from a human sample (blood or PBMCs or bone marrow) based on positive or negative selection. In certain embodiments, the immune cells are cells as reported herein derived from umbilical cord blood, bone marrow, or peripheral blood from human samples.

In certain embodiments, methods comprise the steps of harvesting hematopoietic stem and progenitor cells from the peripheral blood or bone marrow of a subject or a doner. The subject or donor may be treated with one or more clinically approved hematopoietic stem and progenitor cell mobilization agents, for example, Granulocyte-Colony Stimulating Factor (G-CSF), to increase the number of cells that can be collected by apheresis. In certain embodiments, the cancer therapy or CAR therapy is to treat a cancer which is a solid tumor, cellular malignancy, or hematological malignancy. In certain embodiments, the cancer is ependymoma, lung cancer, non-small cell lung cancer, small cell lung cancer, bronchus cancer, mesothelioma, malignant pleural mesothelioma, lung adenocarcinoma, breast cancer, prostate cancer, colon cancer, rectum cancer, colorectal cancer, gastrointestinal cancer, stomach cancer, esophageal cancer, ovarian cancer, cervical cancer, melanoma, kidney cancer, pancreatic cancer, pancreatic ductal adenocarcinoma (PDA), thyroid cancer, brain cancer, glioblastoma (GBM), medulloblastoma, glioma, neuroblastoma, liver cancer, bladder cancer, uterine cancer, bone cancer, osteosarcoma, sarcoma, rhabdomyosarcoma, Ewing's sarcoma, retinoblastoma, nasopharyngeal carcinoma.

### Development of Antigen-Independent Memory Cells After Chronic Viral Infection

The phenotype and fate of stem-like CD8 T cells after the clearance of chronic viral infection was investigated. Experiments were performed to determine whether functional memory CD8 T cells are generated after these cells no longer are receiving TCR stimulation after being stimulated for a long time. In the straight Cl-13 model, virus is cleared in most tissues by about 3 months. LCMV-specific CD8 T cells were characterized in various organs, particularly in the blood and spleen. The cell population TCF1+CD127+CD62L+ was observed similar to the acute LCMV model but not observed in the CD4-depleted Cl-13 infection model. By phenotype this memory subset expressed canonical memory markers but also expressed exhaustion-associated genes such as PD-1, and TOX. This suggests that these cells remembered their past and have imprinted to express genes that are crucial for surviving in the hostile, chronic inflammatory environment. Most notably, this subset was the only subset that expressed CXCR5 and XCL1 which are also only expressed by the stem-like resource CD8 T cells during a chronic infection. The stem-like resource CD8 T cells were transferred into an antigen-free environment to establish the lineage relationship of chronic memory subsets. A stem-like resource subset was able to upregulate CD127, and CD62L and retain its TCF1 expression. These stem-like resource CD8 T cells differentiate into CD62+ stem-like chronic memory cells that can persist without antigen.

Studies were performed to determine the epigenetic landscape of chronic memory subsets compared to the acute memory subsets, and also the product of resting the stem-like resource CD8 T cells in an antigen-free environment. Functional differences of the chronic memory and acute memory were the most surprising. The acute memory and chronic memory CD8 T cell subsets performed similarly after an acute LCMV rechallenge, with a slightly higher numbers in the chronic memory subsets within peripheral tissues. This could be due to the fact that the differentiated effectors derived from chronic memory cells are better at disseminating to peripheral tissues via various chemokine receptors compared to the effectors from an acute memory cells. Even after an acute infection, the chronic memory cells had higher expression of PD-1 and TOX which indicates that these cells remember their past. These chronic memory cells performed superiorly after rechallenge with the chronic LCMV. The TCF1+CD127+CD62L+ chronic memory cells were the only subset that were present at detectable and significant numbers after Cl-13 chronic viral rechallenge. These cells were the most efficient at generating the stem-like resource CD8 T cell during Cl-13 rechallenge of which they are derived. This highlights that the chronic memory cells have been selected to survive after enduring chronic antigenic stimulation for so long in a hostile setting, and the importance of stem-like resource cells in the war against chronic viruses and cancer. Adoptive cell therapies by utilizing these chronic memory cells instead of naïve or acute memory cells are desirable because they have better ability to proliferate, persist, and control the infection or tumors in chronic, and inflammatory settings.

Robust memory of the adaptive immune system is generated after the clearance of an acute infection. These memory T cells persist long term via slow homeostatic proliferation through IL-7 and IL-15 without antigen-stimulation and can rapidly differentiate into effector T cells to quickly control reinfection upon re-stimulation. In contrast, antigen persistence from chronic viral infections and cancer are usually associated with a state of CD8+ T cell dysfunction called exhaustion. Exhausted CD8+ T cells progressively lose their ability to produce important cytokines particularly IL-2, TNFα, and IFNγ and thus are unpoised to control persistent pathogens such as HIV, HBV, and HCV and cancer.

Immunotherapies targeting exhaustion-associated inhibitory receptors such as cytotoxic T-lymphocyte-associated protein 4 (CTLA-4) and programmed cell death 1 (PD-1) have emerged clinically to restore CD8+T cell function for the treatment of non-small cell lung cancer, renal cell carcinoma, metastatic melanoma, Hodgkin's lymphoma, head and neck cancer, and urothelial carcinoma. The emergence of immunotherapies has revolutionized the treatment of cancer.

Immune checkpoint blockade of cytotoxic T-lymphocyte-associated protein 4 (CTLA-4) and programmed cell death 1 (PD-1) present on lymphocytes in the tumor microenvironment are being studies in cancers. New immune checkpoint blockade of other T cell co-inhibitory receptors such as LAG3 and TIM3 are contemplated. Targeting the immune system to treat many different cancers is a promising approach. However some patients are unable to mount a durable CD8 T cell response and the disease progresses. Furthermore, PD-1 blockade alone has shown to induce minimal memory T cell development and re-exhaustion is observed in settings of continued antigen persistence after therapy. Therefore, improvements in current immunotherapies are needed. This is relevant for patients who are living with undetectable disease after chemo- and immunotherapies for cancer, and chronic viral infections such as HBV, HCV, and HIV. An understanding of CD8 T cell regulation in the setting of chronic antigen persistence is crucial to improve therapies that aim to reverse T cell exhaustion and also vaccines against chronic viruses.

PD-1 blockade monotherapy, in combination with TLR7 agonist, or in combination with IL-2 on the LCMV-specific CD8 TCR repertoire is contemplated. PD-1 monotherapy had a no effect on the repertoire of stem-like CD8 T cells but did have a modest effect on the TCR repertoire of the exhausted subset in the spleen but not in the liver. PD-1 blockade alone in the LCMV Clone-13 model has shown not to stably differentiate exhausted T cells into effector and memory cells due to the lack of robust epigenetic reprogramming. Memory T cell development was scant at best and re-exhaustion was inevitable in settings of continued antigen persistence after therapy. Epigenetic reprogramming, specifically DNA methylation, shuts down crucial effector transcriptional programs during exhausted T cell states. Thus in certain embodiments, this disclosure contemplates methods disclosed herein used in combination with transcriptional and epigenetic reprograming.

### CD8 Positive Stem-Like Chronic Memory Cells

Persistent antigenic stimulation during chronic viral infection and cancer results in CD8 T cell dysfunction that is associated with expression of inhibitory receptors such as programmed cell death 1 (PD-1). A better understanding of T cell exhaustion has come from recent studies that have characterized the various T cell states that exist during chronic viral infection and defined the lineage relationships between these different T cell subsets. A subset of PD-1+TCF1+CXCR5+ virus-specific CD8 T cells that act as stem cells to sustain the CD8 T cell response during chronic lymphocytic choriomeningitis virus (LCMV) infection of mice. These LCMV-specific PD-1+ stem-like CD8 T cells maintain their population by a slow self-renewal and also differentiate into more effector like and terminally differentiated CD8 T cells. Thus, these virus-specific PD-1+ stem-like CD8 T cells function as resource cells during chronic infection to keep the virus-specific CD8 T cell response going and in the absence of these PD-1+TCF1+CD8 T cells the LCMV-specific CD8 T cell response wanes in chronically infected mice. Importantly, the rapid proliferative burst of CD8 T cells that is seen after PD-1 blockade comes exclusively from this stem-like CD8 T cell subset that has the ability to proliferate and differentiate into more effector-like T cells.

The PD-1+TCF1+ stemlike CD8 T cell subset is mainly present in the lymphoid organs, particularly in the white pulp of the spleen and lymph nodes, while the terminally differentiated CD8 T cell subset that is derived from the stemlike cells is found in both lymphoid and nonlymphoid organs at sites of viral infection. The quiescent stemlike CD8 T cells do not circulate and are resident in lymphoid tissues, providing a protective niche for their maintenance during chronic infection.

Under conditions of a long-term chronic viral infection with high levels of viremia and systemic infection involving multiple tissues, there are very few virus-specific CD8 T cells in the blood. This is despite the high frequency of virus specific CD8 T cells in both lymphoid and nonlymphoid organs of these chronically infected mice.

The few CD8 T cells that appear in the blood are the more effector-like CD8 T cells that have been recently generated following the proliferation and differentiation of the stem-like CD8 T cells residing in lymphoid organs. It is interesting that PD-1 blockade substantially increases the number of virus-specific CD8 T cells in the blood by acting on the PD-1+ stem-like CD8 T cells and increasing their proliferation and differentiation. Most of the terminally differentiated exhausted CD8 T cells are resident at sites of viral infection in multiple tissues and the stem-like CD8 T cells are resident in lymphoid organs.

Adoptive cell therapy (ACT) utilizes autologous T cells that can be expanded and engineered to recognize target cells (such as cancer) which can lead to disease regression. However, limitations in the persistence of these adoptively transferred T cells, particularly CD8 T cells, have hindered ACT efficacy. The stem-like CD8 T cells that sustain the response during the chronic viral infection persist and differentiate into the stem-like chronic memory cells which have adapted to survive long-term after antigen clearance. In certain embodiments, this disclosure contemplates that PD-1 positive stem-like and stem-like chronic memory CD8 T cells can be isolated and used for adoptive cell therapies for cancer, chronic viral infection, and/or other chronic diseases with better efficacy due to their superior recall potential and persistence. Furthermore, the adoptively transfer of these cells can be paired with PD-1 blockade agents which can significantly bolster the effector differentiation of these transferred cells.

This disclosure relates to CD8 positive stem-like chronic memory cells for uses in managing diseases and conditions associated with T cell exhaustion and compositions related thereto. In certain embodiments, the PD-1 and CD8 positive stem-like chronic memory cells are CD62L positive and CD127 positive. In certain embodiments, the PD-1 and CD8 positive stem-like chronic memory cells may be maintained or replicated in a growth medium.

In certain embodiments, the CD8 positive stem-like chronic memory cells are obtained in a sample from a subject and the cells are isolated from a cell in the sample that express on the surface of the cells CD8, PD1, CD62L, CD44, and CD127.

In certain embodiments, the CD8 positive stem-like chronic memory cells isolated from the sample are cells that express CD8, PD1, CD62L, CD44, and CD127 and are a group of cells that express CD8 on the surface of the cells. In certain embodiments, one can first isolate CD8 positive cells and from the sample providing CD8 positive cells, optionally expanding ex vivo the CD8 positive cells, and isolate cells that are positive for PD1, CD62L, CD44, CD127, or a combination thereof, and thereafter expand ex vivo cells that express CD8, PD1, CD44, CD62L, and CD127.

In certain embodiments, isolating CD8 positive stem-like chronic memory cells or cells from a sample that express CD8, PD1, CD62L, CD44, and CD127 is by isolating cells from lymphoid tissue, thymus gland, spleen, white blood cells, peripheral blood cells, or bone marrow cells that are positive for CD8, PD1, CD62L, CD44, CD127, or combinations thereof.

In certain embodiments, isolating is by positive or negative selection. In certain embodiments, isolating CD8 positive stem-like chronic memory cells or cells from a sample that express CD8, PD1, CD62L, CD44, and CD127 is by mixing the sample with agents that specifically bind independently and individually CD8, PD1, CD62L, CD44, and CD127 and isolating cells by positive selection providing cells positive for CD8, PD1, CD62, CD44, and CD127.

In certain embodiments, isolating CD8 positive stem-like chronic memory cells or cells from a sample that express CD8, PD1, CD62L, CD44, and CD127 is by isolating from a sample cells that express CD8 on the cells providing purified CD8 positive cells; isolating from the sample cells that express PD1 on the cells providing purified PDI and CD8 positive cells; isolating from the purified PD1 and CD8 positive cells, cells that express CD62L on the cells providing purified PD1, CD8, and CD62L positive cells; and isolating from the purified PD1, CD8, and CD62L positive cells, cells that express CD127 on the cells providing purified PD1, CD8, CD62L, CD44, and CD127 positive cells; or combinations thereof.

In certain embodiments, isolating CD8 positive stem-like chronic memory cells or cells from a sample that express CD8, PD1, CD62L, and CD127 is by isolating from a sample cells that express PD1 and CD8 on the cells providing purified CD8 and PDI positive cells and isolating from the purified PD1 and CD8 positive cells, cells that express CD62L on the cells providing purified PD1, CD8, CD44, and CD62L positive cells.

In certain embodiments, isolating CD8 positive stem-like chronic memory cells or cells from a sample that express CD8, PD1, CD62L, and CD127 is by isolating from the sample cells that express PD-1 and CD8 on the cells providing purified PD-1 and CD8 positive cells and isolating from the purified PD-1 and CD8 positive cells, cells that express CD127 on the cells providing purified PD-1, CD8, CD44, and CD127 positive cells.

In certain embodiments, isolating CD8 positive stem-like chronic memory cells or cells from the sample that express CD8, PD1, CD62L, and CD127 is by isolating from the sample cells that express PD-1 on the cells providing purified PD1 positive cells and isolating from the purified PD-1 positive cells, cells that express CD62L on the cells providing purified PD-1 and CD62L positive cells.

In certain embodiments, isolating CD8 positive stem-like chronic memory cells or cells from a sample that express CD8, PD1, CD62L, and CD127 is by isolating from the sample cells that express PD1 on the cells providing purified PD1 positive cells and isolating from the sample cells that express CD127 on the cells providing purified CD127 positive cells; or combinations thereof.

In certain embodiments, for any of the methods disclosed herein, it is contemplated that the method further comprises expanding and/or replicating the isolated cells that are positive for CD8, PD1, CD62L, and CD127 ex vivo providing replicated cells positive for CD8, PD1, CD62L, and CD127.

In certain embodiments, greater than 10%, 20%, 30%, 40%, 50%, 60%, 70%, 80%, 90%, 95%, or 98% percent of total cells are positive for CD8, PD1, CD62L, and CD127.

In certain embodiments, greater than 10%, 20%, 30%, 40%, 50%, 60%, 70%, 80%, 90%, 95%, or 98% percent of total cells are positive for CD8, PD1, CD62L, CD44, and CD127.

In certain embodiments, greater than 10%, 20%, 30%, 40%, 50%, 60%, 70%, 80%, 90%, 95% or 98% percent of total cells are negative for CD4.

In certain embodiments, this disclosure contemplates compositions of cells made by the processes disclosed herein. In certain embodiments, the cells are contained in a growth medium.

In certain embodiments, this disclosure relates to methods of isolating CD8 positive stem-like chronic resource cells comprising, obtaining a sample from a subject, purifying cells in the sample that are PD-1 positive and CD8 positive providing PD1 and CD8 positive cells; purifying cells from the PD-1 and CD8 positive cells providing cells that express TCF1, are CD44 positive, and have no or low expression of Tim3, CD39 negative, or combination of these markers or other markers as disclosed herein, providing isolated CD8 positive stem-like chronic resource cells.

In certain embodiments, the method further comprises the step of expanding the isolated

CD8 positive stem-like chronic resource cells.

In certain embodiments, the method further comprises the step of resting the isolated CD8 positive stem-like chronic resource cells for a sufficient time that expression of CD127 and CD62L is detected.

In certain embodiments, expression of CD127 and CD62L is detected by flow cytometery.

In certain embodiments, resting is in vitro or in vivo.

In certain embodiments, resting is in the absence of T cell receptor agonists, e.g., a cognate peptides, antigen-presenting cells, antibody or small molecule agonists of CD3 and/or T cell receptor.

In certain embodiments, the subject to be treated is the same subject from which the PD1 and CD8 positive cells were originally obtained, or the subject is not the same subject from which the the PD1 and CD8 positive cells were originally obtained.

In certain embodiments, the CD8 positive stem-like chronic memory cells are engineered to express a chimeric antigen receptor.

In certain embodiments, the CD8 positive stem-like chronic memory cells are administered or infused into a subject for use in a medical therapy.

In certain embodiments, the medical therapy is the treatment of cancer, chronic viral infections, or chronic diseases.

In certain embodiments, the CD8 positive stem-like chronic resource cells are administered or infused to a subject in combination with a checkpoint inhibitor.

In certain embodiments, this disclosure relates to CD8 positive stem-like chronic memory cells as disclosed herein expressing a chimeric antigen receptor (CAR). CARs are engineered fusion proteins expressed on cells, e.g., T cells, providing surface receptors that bind to antigens, e.g., tumor associated antigens. The receptor is linked to a transmembrane domain and an endodomain containing a segment that activates T cells signaling. In one example, the receptor domain is a single chain antibody that binds a tumor antigen conjugated to the transmembrane and endodomain.

CARs are typically expressed in cells using an expression vector. The expression vector may be a viral vector capable of infecting the cells or the expression vector may be inserted into the cells by other methods. The CAR typically comprises a transmembrane domain which spans the membrane which is typically a hydrophobic alpha helix. The transmembrane domain may be derived from the CD28 transmembrane domain. Once the expression vector is in the T-cells, a nucleic acid encoding the CAR fusion protein is expressed and the chimeric antigen receptor incorporates into the membrane the cells.

The target binding domain, e.g., single chain antibody, of a CAR may be fused via a spacer to a transmembrane domain and/or to an endodomain which comprises or associates with an intracellular T-cell signaling domain. When the CAR containing cells bind a target cell, e.g., cancer cell, having a targeting domain that is expressed on a target cell, this results in the transmission of an activating signal to the T-cell containing the CAR.

The endodomain is the portion of the CAR involved in signal-transmission. The endodomain either comprises or associates with an intracellular T-cell signaling domain. Although it is not intended that embodiments of this disclosure are limited by any particular mechanism, it is believed that after target bind recognition, receptors cluster and a signal is transmitted to activate the T cell. A commonly used T-cell signaling component is that of CD3-zeta. This transmits an activation signal to the T-cell after the target molecule is bound. In certain embodiments a chimeric CD28 or OX40 can be used with CD3-Zeta to transmit a proliferative/survival signal. The endodomain of the CAR optionally comprises the CD28 endodomain and OX40 and CD3-Zeta endodomain.

In certain embodiments, the CAR comprises a signal peptide so that when the CAR is expressed inside a cell, such as a T-cell, the nascent fusion protein is directed to the endoplasmic reticulum and subsequently incorporates itself to the cell surface. The CAR may comprise a spacer sequence to connect the target binding domain with the transmembrane domain and spatially separate the cell binding domain from the endodomain. A flexible spacer allows to the cell-binding domain to orient in different directions to enable cell binding.

The endodomain sequence may, for example, comprise an IgG1 Fc region, an IgG1 hinge or a CD8 stalk, or a combination thereof. The linker may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

### Methods of Use

In certain embodiments, this disclosure relates to methods of treating cancer, chronic viral infections, or chronic diseases comprising administering to a patient in need thereof an effective amount of CD8 positive stem-like chronic memory cells. In certain embodiments, the CD8 positive stem-like chronic memory cells are derived from the patient and are optionally expanded and/or replicated ex vivo.

In certain embodiments, this disclosure relates to methods of treating cancer comprising administering to a patient in need thereof an effective amount of CD8 positive stem-like chronic memory cells. In certain embodiments, the PD-1 and CD8 positive stem-like chronic memory cells are derived from the patient to be treated, and the cells are isolated, expanded, and/or replicated ex vivo prior to administration.

In certain embodiments, this disclosure relates to methods of treating cancer comprising administering to a patient in need thereof an effective amount of CD8 positive stem-like chronic memory cells wherein the PD-1 and CD8 positive stem-like chronic memory cells are replicated ex vivo prior to administration.

In certain embodiments, the CD8 positive stem-like chronic memory cells or replicated cells thereof are derived from the patient or derived from a person other than the patient. In certain embodiments, the PD-1 and CD8 positive stem-like chronic memory cells or replicated cells thereof are derived from a person other than the patient who recovered from a cancer therapy.

In certain embodiments, the CD8 positive stem-like chronic memory cells comprise a recombinant vector encoding a chimeric antigen receptor.

In certain embodiments, this disclosure relates to methods of treating cancer, neuroblastoma, or ganglioneuroblastoma comprising administering to a subject in need thereof an effective of CD8 positive stem-like chronic memory cells expressing a chimeric antigen receptor with a targeting domine that specifically binds CD171.

In certain embodiments, this disclosure relates to methods of treating cancer such as adenocarcinoma, colorectal cancer, breast cancer, or liver cancer comprising administering to a subject in need thereof an effective of CD8 positive stem-like chronic memory cells expressing a chimeric antigen receptor with a targeting domine that specifically binds CEA (carcinoembryonic antigen).

In certain embodiments, this disclosure relates to methods of treating cancer such as glioblastoma comprising administering to a subject in need thereof an effective of CD8 positive stem-like chronic memory cells expressing a chimeric antigen receptor with a targeting domine that specifically binds epidermal growth factor receptor variant III (EGFRvIII).

In certain embodiments, this disclosure relates to methods of treating cancer, glioblastoma, or glioma comprising administering to a subject in need thereof an effective of CD8 positive stem-like chronic memory cells expressing a chimeric antigen receptor with a targeting domine that specifically binds epidermal growth factor receptor variant (EGRF).

In certain embodiments, this disclosure relates to methods of treating cancer such as ovarian cancer comprising administering to a subject in need thereof an effective of CD8 positive stem-like chronic memory cells expressing a chimeric antigen receptor with a targeting domine that specifically binds follicle stimulating hormone receptor (FSHR).

In certain embodiments, this disclosure relates to methods of treating cancer or neuroblastoma comprising administering to a subject in need thereof an effective of CD8 positive stem-like chronic memory cells expressing a chimeric antigen receptor with a targeting domine that specifically binds neuroblastoma disialoganglioside (GD2).

In certain embodiments, this disclosure relates to methods of treating cancer such as hepatocellular carcinoma comprising administering to a subject in need thereof an effective of CD8 positive stem-like chronic memory cells expressing a chimeric antigen receptor with a targeting domine that specifically binds glypican-3 (GPC3).

In certain embodiments, this disclosure relates to methods of treating cancer such as lung cancer comprising administering to a subject in need thereof an effective of CD8 positive stem-like chronic memory cells expressing a chimeric antigen receptor with a targeting domine that specifically binds human epidermal growth factor receptor 2 (HER2).

In certain embodiments, this disclosure relates to methods of treating cancer such as glioblastoma comprising administering to a subject in need thereof an effective of CD8 positive stem-like chronic memory cells expressing a chimeric antigen receptor with a targeting domine that specifically binds IL-13 receptor a2 (IL 13Ra2).

In certain embodiments, this disclosure relates to methods of treating cancer or prostate cancer comprising administering to a subject in need thereof an effective of CD8 positive stem-like chronic memory cells expressing a chimeric antigen receptor with a targeting domine that specifically binds prostate specific membrane antigen (PSMA).

In certain embodiments, this disclosure relates to methods of treating cancer such as pancreatic cancer or ovarian cancer comprising administering to a subject in need thereof an effective of CD8 positive stem-like chronic memory cells expressing a chimeric antigen receptor with a targeting domine that specifically binds mesothelin.

In certain embodiments, the CD8 positive stem-like chronic memory cells are administered in combination a checkpoint inhibitor. In certain embodiments, the checkpoint inhibitor is an anti-PD1 antibody or anti-PD-L1 antibody. In certain embodiments, the checkpoint inhibitor is an anti-PD1 antibody or anti-PD-L1 antibody is selected from pembrolizumab, nivolumab, cemiplimab, atezolizumab, dostarlimab, durvalumab, and avelumab.

In certain embodiments, the cancer is basal cell carcinoma, bladder cancer, breast cancer, cervical cancer, colorectal cancer, endometrial cancer, esophageal carcinoma, gastric cancer, head and neck cancer, hepatocellular carcinoma, Hodgkin's lymphoma, malignant pleural mesothelioma, melanoma, Merkel cell carcinoma, lung cancer, small cell lung cancer, non-small cell cancer, lymphoma, renal cell carcinoma, solid tumors, squamous cell carcinoma, stomach cancer, or urothelial carcinoma.

In certain embodiments, this disclosure relates to methods of treating chronic viral infection comprising administering to a subject in need thereof an effective amount of CD8 positive stem-like chronic memory cells. In certain embodiments, the chronic viral infection is selected from HBV, HCV, and HIV. In certain embodiments, the composition of cells is administered in combination with another antiviral agent.

In certain embodiments, the CD8 positive stem-like chronic memory cells are CD62L positive and CD127 positive. In certain embodiments, the CD8 positive stem-like chronic memory cells are replicated ex vivo prior to administration. In certain embodiments, the PD-1 and CD8 positive stem-like chronic memory cells or replicated cells thereof are derived from the patient or derived from a person other than the patient. In certain embodiments, the PD-1 and CD8 positive stem-like chronic memory cells or replicated cells thereof are derived from a person other than the patient who recovered from an anti-viral therapy.

In certain embodiments, this disclosure relates to methods of treating chronic disease comprising administering to a subject in need thereof an effective amount of CD8 positive stem-like chronic memory cells.

In certain embodiments, the CD8 positive stem-like chronic memory cells or replicated cells thereof are derived from the patient or derived from a person other than the patient. In certain embodiments, the PD-1 and CD8 positive stem-like chronic memory cells or replicated cells thereof are derived from a person other than the patient who recovered from a viral infection.

In certain embodiments, the CD8 positive stem-like chronic memory cells or replicated cells thereof are derived from the patient or derived from a person other than the patient. In certain embodiments, the PD-1 and CD8 positive stem-like chronic memory cells or replicated cells thereof are derived from a person other than the patient who received a vaccination.

In certain embodiments, the CD8 positive stem-like chronic memory cells are CD62L positive, CD44 positive, CD127 positive, or combination thereof.

In certain embodiments, this disclosure relates to methods of reducing or eliminating expression of one or more genes required for the induction and/or maintenance of stem-like chronic memory CD8 T cells.

In certain embodiments, this disclosure relates to methods of increasing or inducing expression of one or more genes required for the induction and/or maintenance of stem-like chronic memory CD8 T cells.

In certain embodiments, increasing or inducing expression of one or more genes required for the induction and/or maintenance of stem-like chronic memory CD8 T cells or reducing or eliminating expression of one or more genes required for the induction and/or maintenance of stem-like chronic memory CD8 T cells is by a method selected from the group consisting of RNA interference, clustered interspersed short palindromic repeat (CRISPR)/CRISPR-associated protein (Cas) system, meganucleases, transcription activator like effector nucleases (TALENs), Zinc-finger nucleases (ZFNs), antisense, ribozymes and CRISPR inhibition system comprising dead Cas9.

In certain embodiments, increasing or inducing gene expression or reducing or eliminating gene expression is a gene selected from Serpina3g, Klre1, Klrc1, Cd38, Pdcd1, Anxa2, Prr51, Dgkh, Cxcr5, Eomes, Klrg1, Tceal9, Bex3, Qpct, Lmna, Ldhb, Rnf130, Gm2a, Acot7, Racgap1, Wfikkn2, Plscr4, Xcl1, Tox, Slc2a3, Ogfrl1, Satb1, Tmem51, Serpina3f, Nr3c2, Casp1, Fcgr2b, Myadm, Gzmk, Pros1, Nkg7, Osbpl3, Fgl2, Sesn1, Cpne7, Samd3, Aplp1, Vmp1, Ssh1, Ikzf3, Maf, Pygl, Tnfsf13b, Tacc1, Cldnd1, BC064078, Cd8b1, Lgals1, Tmem154, Tigit, Gimap7, Plscr1, Kcnip3, Ms4a4a, Ppp2r2c, Cyp4f16, Asb2, Ttyh3, Ptpn11, Ildr1, Radx, Slpr5, Ppplr11, Rapgef6, Acadl, Lpin1, Lgals3, Lratd1, I17, Atp6v0d2, 2310001H17Rik, Slc27a4, Tle1, Furin, Trim2, Pctp, Iigp1, Rasl12, Armc7, Nsmaf, Metrnl, Tmbim4, Cish, Pvrig, Mlf1, Sytl1, Zbtb32, Itgb2, Sqle, Tppp3, Dtx4, Srebf2, Klf10, Lmo1, Abcg2, Atplal, Ptpn6, Peli1, Litaf, Stx11, Tanc1, S100a6, Lrrk1, Itgax, Ybx3, Vwa5a, Sh2dla, Kcnn4, Gas7, Rnf128, Vim, Tmem171, S100a11, Cmtm6, Cd82, Nfatc3, Hif1a, 9630013K17Rik, Akrle1, Pde3b, Tspan3, Chst12, Pea15a, Snx9, Rbm47, Tbcld2, Tnfsf4, Sidt1, Ywhah, Klrk1, Tpd52, Ctsd, Prr13, Nr4a2, Sulf2, Crip2, Map3k3, Stim1, Fcgrt, Dact2, B630019K06Rik, Mapk12, Ahcy, Pacs2, Fam241a, Gnb4, Cyp2s1, Pdk1, Klrb1b, Ica1, Dleu2, Stk39, Cmah, B4galt4, Cd401g, Tbl1xr1, Cpm, Hic1, Tmem159, Bhlhe40, Foxn3, Cyth3, Mrtfa, Zc2hcla, Cd72, Emp3, B3gnt7, Scamp3, Atp11b, Cenpj, NA, Bex2, Gm4208, Scly, Ncoa3, Zcchc18, Naip5, Synel, Il12rb2, Gm35037, Pls3, Osr2, Rnf19b, Arsb, Btg2, Myolg, Xylt1, Efhd2, Gm44175, Gent1, Ly6a2, Kif5c, Mcub, Galnt10, Itgb3, Pde4b, Stat5b, Klrb1c, Srgap2, Hspa2, AU020206, Dop1a, Wtap, Plac8, Cd79b, Pdgfb, Wls, Cdk4, Tppp, Cd22, Psen2, Sipa 1l1, Tlr4, Galm, Gm 15228, Stk38, Batf, Gm52993, Gpr87, Sh3yl1, Sgce, Epha3, Ccdc92b, Ankrd13a, Oas1a, Atg3, Ywhaq, Inpp5f, Ncf4, Smap1, Rin3, Tbc1d1, Ryr1, Rictor, Gzmb, Bbs9, Hlcs, Adrb1, Prmt2, Zfp512, Ociad2, Gm11454, Jpt1, Alox8, Gm35363, H2-Q5, Gpr15, Gm8817, Stard3nl, Car5b, Il2rb, Pak6, Pafah1b3, Crlf3, Ucp2, Pfkp, Txnl, Epn2, Nin, Pax9, Dapk2, Cd86, Gpr18, Ccnd2, Rapgef2, Fbxw11, Clybl, Add3, Cd200r4, Bicd1, Slc25a24, Polr2e, H2-Q7, Dgkd, Cdc42se2, Ar, Gm 10522, Clqtnf6, Trib3, Cit, Cpq, Pik3cd, Tubgcp6, Bcl91, Arf6, Serp2, App, Reep5, 1700017B05Rik, Agpat2, Mzf1, Grhl2, Ctdsp2, Tmem231, Tmem71, Frmd6, Xbp1, Gm15987, Cers4, Stk4, Tespa1, Ctss, Rasgrp1, Exosc8, Pfkfb4, Gem, Septin4, Gm28053, 6330403K07Rik, Rab32, Smg6, Cflar, Tanc2, Mrpl38, Cyp17a1, Ran, Agrn, Gm14125, Icos, Cnot61, H2-Ob, Tent5a, Itgam, Ranbp10, Gca, Jtb, Tob1, Sypl, Lilr4b, Ube216, Pwwp2b, Ralb, H2az1, Tafa3, Pqlc3, Jak3, Lamc1, Gss, Nr2f6, Fyn, Ift140, Rasgef1a, Slamf1, Cxcr3, Dnajc2, Prkcb, Pls1, Zeb2, Fam3b, Myc, Acyl, Ndfip1, Cd160, GOs2, Sbf1, Slc25a13, Gm4841, Dpp4, Zmat1, Tnfrsf8, Stmn1, Mlec, Slc25a46, Dtx1, Orai2, Scart1, Agpat4, Phf3, Ighm, Plxna1, Enpp5, Crip1, Cd9, Gbp11, Septin11, Tmem131l, Klh14, Pdia6, Cd47, Edem1, Calm1, Apbb1, Specc1, Eif4g3, Pkp4, Hgfac, Selenow, Prom1, Ap3m2, Gm5127, Tmem229b, Il10rb, Rnaseh2c, Card6, Ephb6, Lrrc8c, Rab37, Tex2, Id3, Cela1, Puf60, Sla2, Siahla, Chd4, Nab2, Psmb8, Baiap3, Ranbp1, Ube2g2, Gpr183, Spc25, Coro2a, Dyrk3, Calcrl, Apol10b, Il10ra, Ddc, Gm26740, Kbtbd3, Eif4ebp2, Zfp654, Ylpm1, Galnt6, Apc, Rnf166, Gm371, Tmed7, Als2cl, Frrs1, Hk2, Klf7, Arhgef18, Mast3, Tmem205, Rtn4rl1, Pdia4, Gm53056, Kifc3, Trim14, Actn1, Bptf, Zdhhc17, Gm15518, 2610507B11Rik, Ech1, Ipcef1, Usp40, 1700001022Rik, Zfp646, Vamp8, Prss2, Napsa, Susd3, Igkc, Rab3a, Slc25a15, Gm44699, Il4ra, Frmd4a, Zc3h6, Fam 168b, Dnajc15, Tnfrsf13c, Akrlc13, Ttc17, Tbcel, Fer115, Snx4, Srsf9, Kcnc1, Gm44423, Suco, Nup85, Clint1, Ctla2a, Arid1b, Manla2, Ipo11, Ttn, Usp18, Il17ra, Gnpat, Dennd3, Rubcnl, Tpm4, Ppcs, Card19, Dhrs7, Rasgef1b, Fnta, Fmnl3, Gbp3, Smg1, D630039A03Rik, Arl11, Sfr1, Sidt2, Ifit2, Ifit3, Gm527, H2-Q6, Fam114a1, Kansl1, Sh3bp5, Zfpl1, Fmrl, Pearl, Chst2, Acp5, Epsti1, Ly6c2, Sart3, Smpdl3a, Hexa, Gpr55, Dram1, Map4k2, Ctsc, Glol, Lrfnl, Gimap4, Pdlim1, Insl6, Zfp106, Sipa1, Nuak2, Tmem237, Arhgap1, BC147527, Nedd9, 2510009E07Rik, Cox 7c, Sertad3, 2410022M11Rik, Susd6, Tmbim1, Cx3cr1, Pts, Ccr7, Plpp1, Adgrg5, Trak2, Gm53055, Snrnp200, Errfi1, Herc1, Gm49703, Gm32772, Zfp292, Zfp518a, Gm15912, Etv3, F730043M19Rik, Zfp445, Gmfb, Pou4f1, Lcmt2, Ugcg, Rnf167, Spry2, Nab1, Ppplr12a, Tbx6, Gm38130, N4bp2, Rnf181, Cnr2, Clec21, Lmf1, Fam78a, Etnk1, Cerk, Unc93b1, Nr4al, Ptger3, Cd226, Gpr155, Mtg2, Pvr, Ccl3, Kcnj8, Ubn1, Anp32b, Elmo1, Gm43011, Notch1, Pacsin2, Cst3, Mrpl41, Ikbke, Gm30948, Txnrd1, Hdac7, Gm15503, Panx1, Mrgbp, Man2a2, Aplg1, Ubb, Col23a1, Atpov1g1, Ryk, Serpinbla, Pik3r5, Dgka, Klri2, Trp53i13, Zfp422, Adgrb2, Trim7, Ankrd44, Tusc1, 2810429I04Rik, Lncbate6, 1810037I17Rik, Polr3f, Nrarp, Stambpl1, Fntb, B4galt7, Ubxn4, AA467197, Bbx, Foxp1, Ssh2, Fam174b, Zfp239, Smyd1, Ubac2, Gpaa1, Smad1, Pitpnm2, Dmtf1, Gvin-ps7, Serpinb6a, Spic, Zfp318, Lrrfip2, Nap113, Trappc8, Agps, Nhsl2, Bmx, Gm19589, Prkx, Cnot1, Ulk1, Siah2, Myl4, Vipr1, Larp4b, Dsel, Tpst2, Map3k2, Ar15c, Ikzf1, Pcx, Itk, Ndufaf4, Ifi2712a, Abi2, Zfp322a, Plod2, Gm17435, Fndc3a, Tec, Rps6ka1, Smtn, Plekhg2, Chd7, Bscl2, Ndel, Epb4113, Kremen1, Piga, Itgad, Ptprk, Irf2, Kctd17, Abca7, Rbm33, Ip6k2, Gm42495, Xpo1, Tgfbr2, Hmgal, Phf11b, Thada, Plcl1, Emb, Cyb5dl, Atpif1, Focad, Kdm6a, Tm9sf3, Snx6, Tnfrsf13b, Erp44, B4galt1, Cd37, Lax1, Kdm5a, Zfp120, Rab3d, Slc43a2, Asf1b, Extl3, Crtc3, Insig1, Naa15, Pena, Lcn4, Tmem 127, Cpox, Sec16a, Tspyl2, Pacs1, Cdkn2c, 9930111J21Rik1, Zfp652, Dpy30, Gm45191, Madd, Zdhhc8, Bnip3, Pricklel, Lamp1, Fcho1, Cdkn2b, Gm48585, Vapa, Thap12, Cdc25a, Slamf7, Ccdc38, Pum2, Prom2, Dnajb11, Cnp, Trabd, Gm48138, Enpp2, Usp28, Rnf7, Gm6967, Gm28100, S100a4, Ptk2b, Dkk11, Ext1, Mal, Armcx2, Il18rap, Actn2, Ptprcap, Myd88, Aqp9, Cdk 19, Tpbgl, Fut8, Nup153, Paqr4, Cnot6, Zbtb1, Rnf126, P2ryl2, Arfgap3, Utp11, Smpdl3b, Rbsn, Gm7265, Cul3, Fkbp10, Pak2, Phospho2, Sin3a, Abcc5, Hivep3, Elovl1, Dock10, Prdm9, Mbnl2, Cox7a2, Crmp1, Cipc, Asap2, Setbp1, Wdr48, Tusc2, Pkm, Gm44321, Dck, Inip, Klh125, Fan1, Stc2, Klrb1f, Socs3, S1pr4, Dusp2, Spsb1, Epb41, Gm1826, Cmtm7, Ssx2ip, Mdm4, Zdhhc22, Cst7, St6galnac6, Setd1a, Arl15, Oplah, Gm11342, Themis, Gm35035, Med16, Afdn, Mast2, Timp2, Zfp597, Rreb1, Faap 100, Anxa5, Tug1, Bahd1, Sec24c, Arl4a, Iglc2, Ergic2, Spock2, Optn, Tmsb4x, Dipk1b, Srsf7, Srp72, Crebbp, Gigyf2, Yiflb, Smpd5, Mrps15, 4932438A13Rik, Id2, Zfp182, Casp4, Prdm16, Cdc42ep3, Nsmf, Lrrc28, Elovl4, Phlda3, Hnrnpl, Farp1, Blm, Rexo2, Cdc42ep4, Fam 169b, Dcaf12, Gm4956, Tradd, Mllt1, Gm37248, Mid2, Klh122, Tmem 184c, Gm8013, Glplr, Ubxn7, Tmem106a, Uri1, Gm27162, Ifi206, Rpa2, Cracr2a, Polr3b, Grap2, Cisd3, Zmym3, Lockd, Ube3b, Lrig1, Scmh1, 1700010I14Rik, Acoxl, Rftn2, Car12, Qrfp, Cacna2d4, Tfpi, Tbc1d19, Stx3, Bcl2111, 2900005J15Rik, Gm6934, Efcab2, C230085N15Rik, Prx12a, 1110032A03Rik, Echdc1, Plscr3, Spin2c, Slamf6, Bphl, Mettl15, Tmem9, Oasl2, Traf5, Tmem141, Abhd14a, Abcb8, Rgmb, Zfp202, Gm10275, Pus71, Rnf157, Unc5a, Trib2, Heatr5a, Tefm, Scarb1, Ccdc102a, Apex1, or combinations thereof.

### Compositions and Kits

In certain embodiments, this disclosure relates to composition made by the processes provided described herein. In certain embodiments, the cells disclosed herein or made by processes disclosed herein may be maintained or replicated in a growth medium.

In certain embodiments, this disclosure relates to kits or articles of manufacture comprising cells or compositions made by the processes provided herein and instructions for use by, e.g., a healthcare professional or patient. The kits or articles of manufacture are a vial, syringe, canula, or other transfer device containing cells as described herein.

Preferably, the vial, syringe, canula, or other transfer device is composed of glass, plastic, metal, or a polymeric material chosen from a cyclic olefin polymer or copolymer. The syringe, ampoule, cartridge, or vial can be manufactured of any suitable material, such as glass or plastic and may include rubber materials, such as rubber stoppers for vials and rubber plungers and rubber seals for syringes and cartridges. In certain embodiments, the kit may further comprise instructions for use and/or a clinical package leaflet. In any embodiment of the products as defined herein, this disclosure also encompasses the presence of packaging material, instructions for use, and/or clinical package leaflets, e.g., as required by regulatory aspects.

### Isolation and Utilization of Stem-Like Chronic Memory Cells for Adoptive Cell Therapy

CD8 T cells play a vital role in homeostasis by recognizing their cognate antigen and eliminating their target such as in the case of cancerous and virally infected cells. If the antigenic stimulus is cleared, as in an acute viral infection, a subset of the heterogenous pool of effector CD8 T cells will survive to become long-lived memory cells that are longitudinally maintained believed to be independent of TCR stimulation. In contrast, T cells that endure persistent antigenic stimulation induced by chronic viral infection or cancer eventually become dysfunctional. CD8 T cell present in these chronic settings are associated with the upregulation of various inhibitory receptors, most notably programmed cell death 1 (PD-1), and thus have the subsequent inability to completely clear the pathogen or cancer due to functional impairments. Data reported herein indicates that chronic memory stem-like CD8 T cells maintain TCF1 expression and upregulated CD127 and CD62L. Markers that define the stem-like chronic memory cells include PD-1+CD127+CD62L+CCR7+TIM3−TCF1+ TOX+. Functionally, the chronic memory cells had superior proliferation, persistence, and effector potential against rechallenge with a chronic virus.

Although it is not intended that certain embodiments of this disclosures be limited by any particular mechanism, it is believed that at least two distinct populations, in regard to their gene expression profiles, proliferative potential, and dysfunctional states, exist in chronic antigen settings. One subset, referred to as the stem-like CD8 T cells, resides in the T cell zone of lymphoid tissues and have the capacity to self-renew and persist in highly inflammatory environments. The stem-like cells differentiate into the second population which harbor effector function, such as granzyme B, but are limited in their proliferative and survival potential. The slow self-renewal, and differentiation of stem-like cells into effectors are important aspect of cancer immunotherapy efficacy, particularly ones targeting PD-1, and overall prognosis of cancer patients.

It is not known exactly how stem-like CD8 T cell are regulated and maintained after the clearance of chronic antigen stimulation, e.g., it is not known whether memory CD8 T cells emerge similar to acute memory or what is their phenotype and function. This is relevant for patients who are living with undetectable disease after various treatments for cancers, and chronic viral infections such as HBV, HCV, and HIV. An understanding of CD8 T cell regulation in the setting of chronic antigen persistence is important to improve therapies that aim to reverse T cell exhaustion, to vaccinate against chronic viruses and cancer, and to engineer cells for adoptive cell therapy.

Stem-like resource cells have been identified as important for sustaining CD8 T cell responses during human chronic viral infections, cancer, and autoimmunity. Furthermore, these cells are targets of PD-1 blockade by providing the proliferative burst necessary to control the tumors. When stem-like resource cells are isolated during a state of chronic TCR stimulation (i.e. chronic viral infection) then transferred into a setting without TCR stimulation, they differentiate into stem-like chronic memory cells by upregulating CD127, CD62L while maintaining expression of TCF1, PD-1, and TOX. The rested stem-like resource cells that have differentiated into stem-like chronic memory cells after cessation of TCR stimulation can then be utilized for adoptive cell therapy.

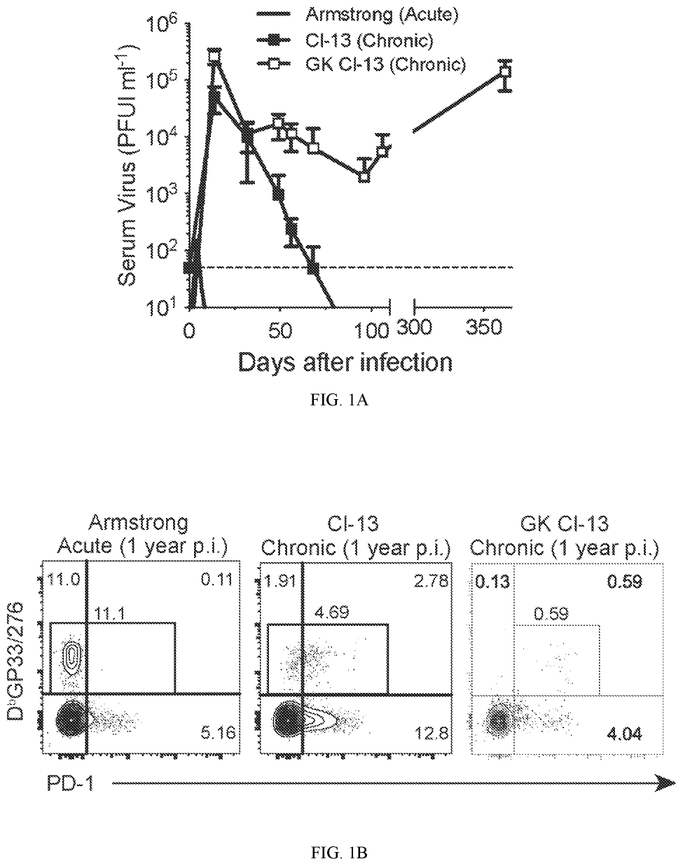

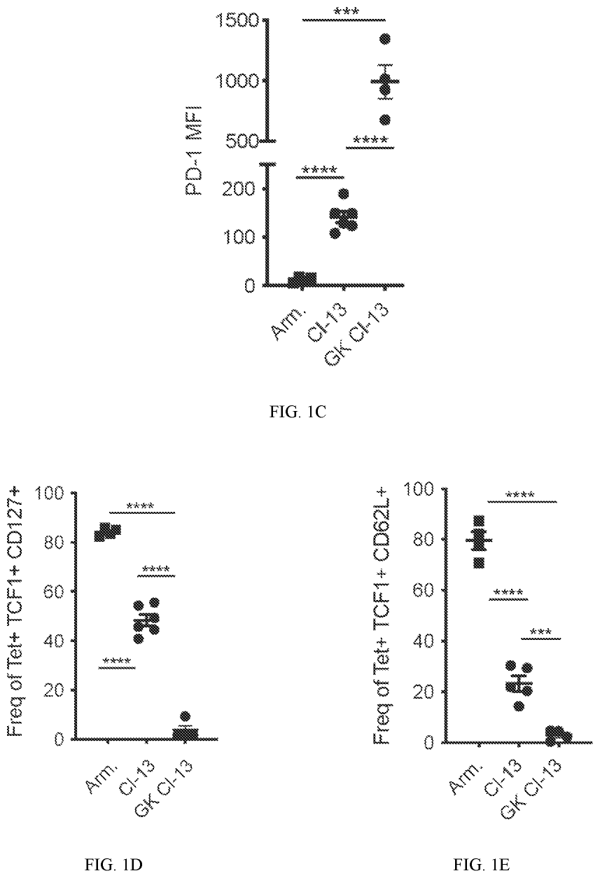

To investigate the phenotype of CD8 T cells after the clearance of chronic antigen stimulation, murine acute and chronic lymphocytic choriomeningitis virus (LCMV) infection models were used (FIG. 1A). The acute strain of LCMV is quickly cleared and robust memory CD8 T cells are generated after clearance. The chronic LCMV infections are more prolonged, lasting several months. The phenotype of LCMV-specific CD8 T cells after the clearance of the acute and chronic strains were assessed after 1 years post-infection. Greater than one year after the clearance of LCMV Armstrong and Cl-13 infections, antigen-specific CD8 T cells persist in various organs of mice, specifically the spleen (FIG. 1B). Interestingly, the tetramer positive cells derived from Cl-13 chronically infected mice maintain expression of PD-1 despite the undetectable viral burden in the blood (FIG. 1A-B). Similar findings were observed in HCV patients where persistent CD8 T cells maintain PD-1 expression after HCV clearance. Antigen-specific CD8 T cells in chronically infected mice were dichotomously expressing CD62L similar to central and effector memory subsets seen in the acute infection model and upregulated the IL-7 receptor (CD127) (FIG. 1D). Antigen-specific cells were then sorted based on CD62L protein expression in both Armstrong and Cl-13 infected mice.

### Unique Gene Expression Signatures are Observed Between Acute and Chronic Memory Subsets

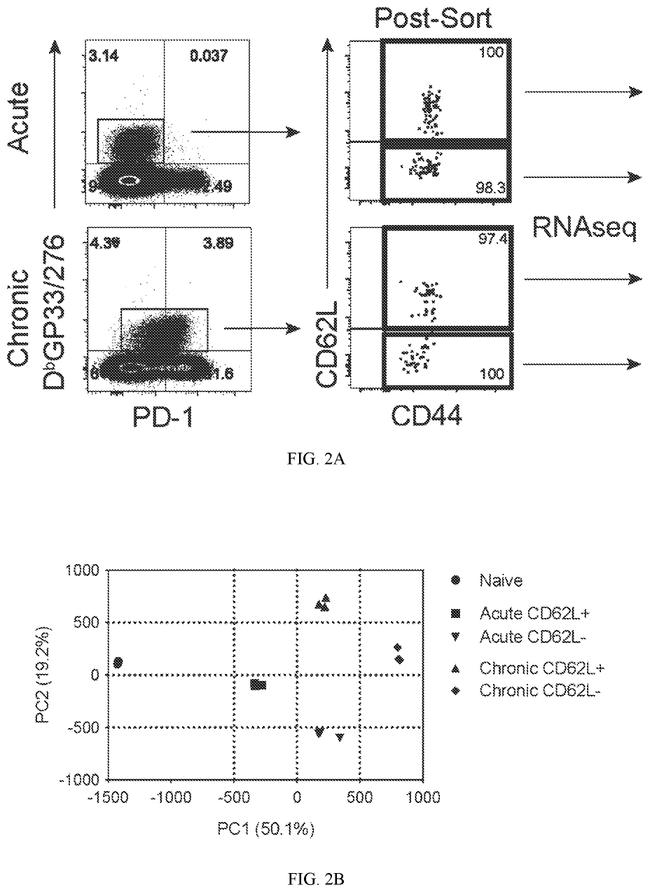

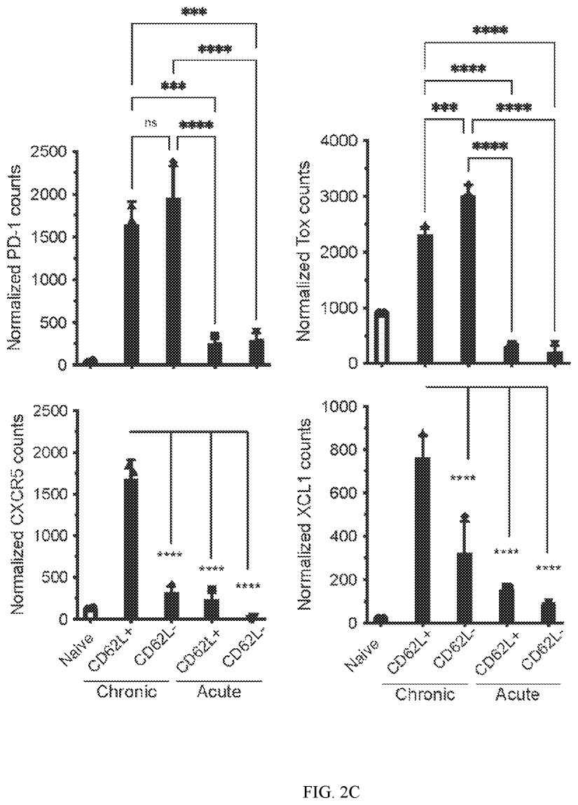

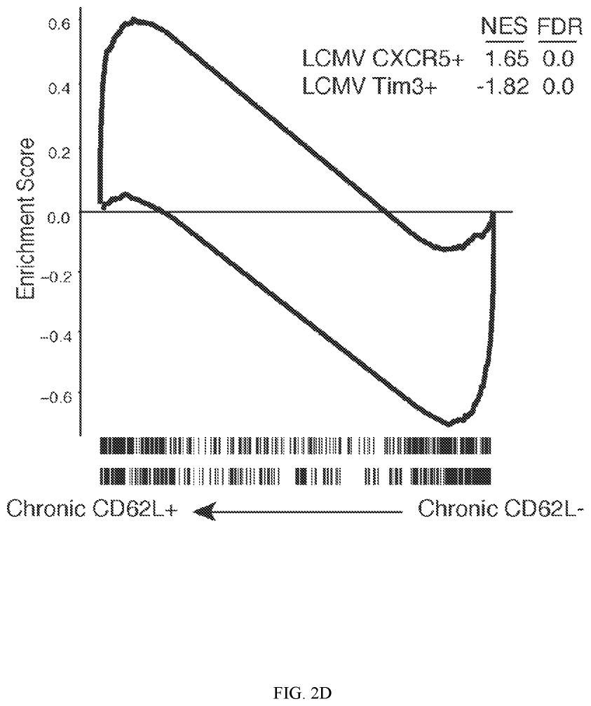

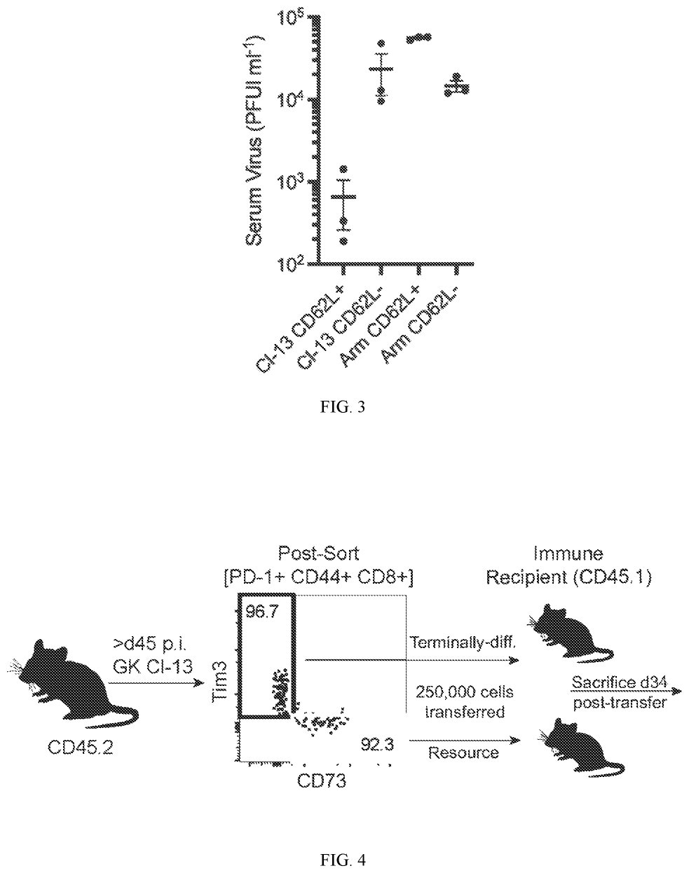

The transcriptomics of CD62L+ and CD62L− subsets were generated after an acute and chronic LCMV infections. Antigen-specific cells were sorted based on CD44 and CD62L protein expression in both Armstrong and Cl-13 cleared mice greater than one year after infection and RNA-seq analyses were performed on these subsets (FIG. 2A). PCA analysis revealed that each subset is transcriptionally distinct from one another (FIG. 2B). Notably, certain inhibitory receptors (PD-1, CD101, TIGIT, and CD160) were highly expressed in the Cl-13 experienced cells. Interestingly, CTLA4, 2B4, Tim3, and LAG3 were only highly expressed in the CD62L− chronic memory cells highlighting their similarity with the CXCR5−Tim3+ terminally differentiated exhausted cells founding during a chronic LCMV infection. Many of the effector molecules such as GzmB were primarily expressed only in the CD62L− subsets of acute and chronic memory cells while GzmM was highly expressed in the CD62L+ memory subsets. GzmK was unique in that it was expressed highly exclusively in the Cl-13 experienced cells. All subsets expressed TCF1/7, and ID3 transcription factors but at a lower level compared to uninfected naïve CD8 T cells. Intriguingly, exhaustion associated transcription factors, TOX, EOMES, MAF, BATF, were expressed solely in the Cl-13 cleared cells. These transcription factors especially TOX may be playing an important role in the stability of epigenetic remodeling seen in exhausted CD8 T cells even after the antigenic stimulation is ceased. Transcription factors associated with effector function and terminal differentiation such as TBET, ID2, and BLIMP1 were highly expressed in the CD62L memory subsets. The most striking difference was in chemokine and chemokine receptors. CXCR5 and XCL1 which are only expressed in the stem-like resource cells were expressed at the highest level in only the CD62L+ chronic memory cells. Consistent with the sorting strategy, PD-1 (Pdcd1) and Tox mRNA expression levels were the highest in the Cl-13 infected cells especially the CD62L− subset of chronic memory cells (FIG. 2C). Interestingly, the Cl-13 cleared CD62L+ cells had the highest expression of Cxcr5, Xcl1, and Tcf7 similar to the stem-like CD8 T cells (FIG. 2C); Cl-13 CD62L− cells, however, did not produce these transcripts but had high levels of Haver2 (Tim3), Cd244 (2B4), and Gzmb characteristic of the exhausted CD8 T cell subsets. Because these chronic memory subset had such striking similarities between the CXCR5+Tim3− stem-like resource and the CXCR5−Tim3+ exhausted CD8 T cells, gene set enrichment analysis was performed to quantify their transcriptomic similarities. GSEA revealed that the CD62L+ chronic memory cells were the most similar to the CXCR5+Tim3− stem-like resource while the CD62L− chronic memory cells were the most similar to the CXCR5−Tim3+ exhausted CD8 T cells (FIG. 2D). Taken together, each subset of memory cells are transcriptionally distinct and chronic memory subsets resemble the stem-like resource and the terminally-exhausted CD8 T cells found during chronic viral infections and cancer.

### Differentiation of Stem-Like CD8 T Cells into Chronic Memory Cells After Antigen Withdrawal

From the RNA sequencing analyses, it was hypothesized that the stem-like and terminally differentiated CD8 T cells are differentiating into distinct memory subsets after antigen clearance. The lineage relationship and the origin of these persistent T cell subsets found after the clearance of chronic LCMV infection was examined. Mice were infected with the chronic LCMV in the CD4-depleted model where the stem-like and terminally-differentiated subsets are generated in a distinct manner. After viremia reaches homeostasis, circa >45 days p.i., stem-like resource (PD-1+CD44+Tim3−CD73+CD39−) and terminally-differentiated (PD-1+CD44+Tim3+CD73−CD39+) CD8 T cells were sorted and equal numbers of cells were transferred into congenically distinct LCMV immune recipient mice (FIG. 4). It is important to transfer these cells into an LCMV-immune mice because transfer of Cl-13 into the recipient can be neutralized quickly to establish a truly antigen-free environment to study the lineage relationship of chronic memory cells after chronic antigen stimulation. Persistence and phenotype were assessed around 30 days post-transfer. Equal numbers of total cells were transferred but because frequencies of GP33+ and GP276+ cell are different between the two subset numbers of donor cells post-transfer were normalized. After normalization, persistence of GP33+ and GP276+ resource cells were around 10-fold greater than that of the terminally-differentiated donors (FIG. 4B).

The phenotype changes of GP33+ GP276+ donors were assessed after antigen withdrawal. TCF1 expression did not change with antigen withdrawal: stem-like resource cells maintained TCF1 expression and the terminally-differentiated cells remained TCFI(negative). PD-1 expression decreased in both donor subsets but they both remained PD-1lo similar to the levels found in Cl-13 cleared memory cells. The resource cells remained Tim3−CD73+ while the terminally-differentiated donors downregulated Tim3 and upregulated CD73. As for the canonical memory markers, CD127 and CD62L, the terminally-differentiated donors could not upregulate these functionally important molecules while the majority of the resource cells upregulated CD127 and started to upregulate CD62L. Interestingly, the terminally-differentiated donors remained CD69+ but the resource cells downregulated CD69 suggesting a shift towards migratory potential. Finally, GzmB expression remained the same in both donor populations. This experiment indicates that the stem-like CD8 T cells have better capacity to persist in an antigen-free environment and remain TCF1+ and are able to more efficiently upregulate IL7 receptor and L-selectin (CD62L) which are characteristic molecules expressed by naïve and memory CD8 T cells.

### CD62L+ Chronic Memory Subset has Similar Recall Potential as the Acute Central Memory Subset Against an Acute Infection

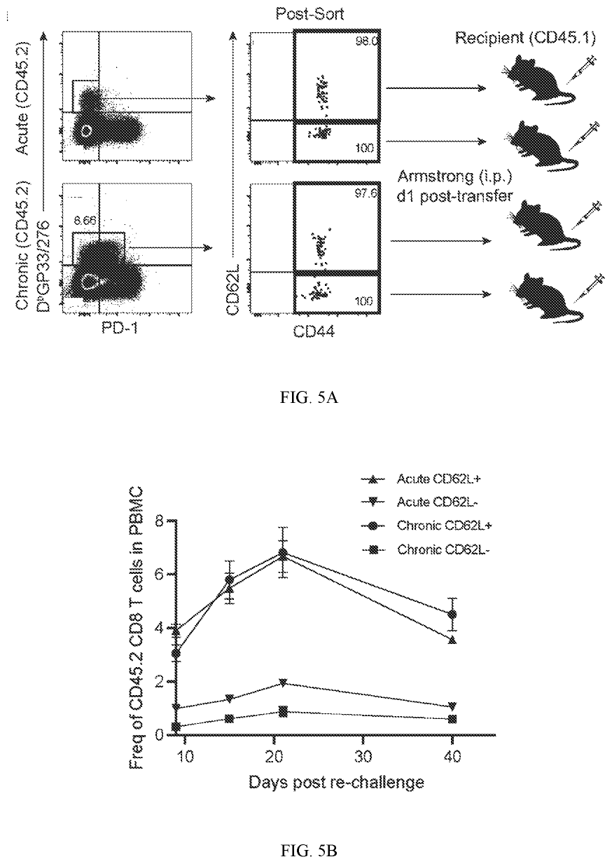

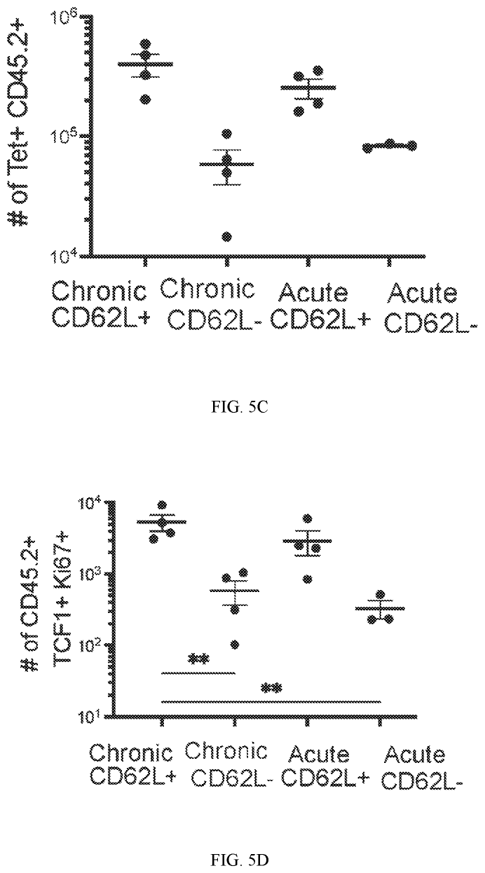

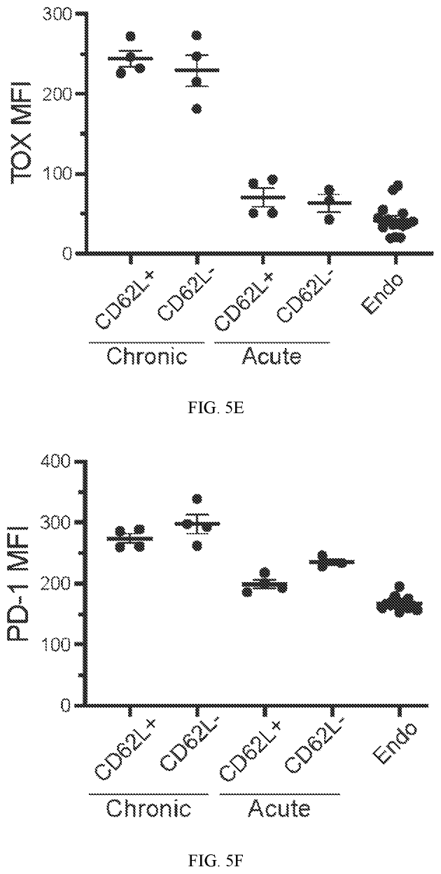

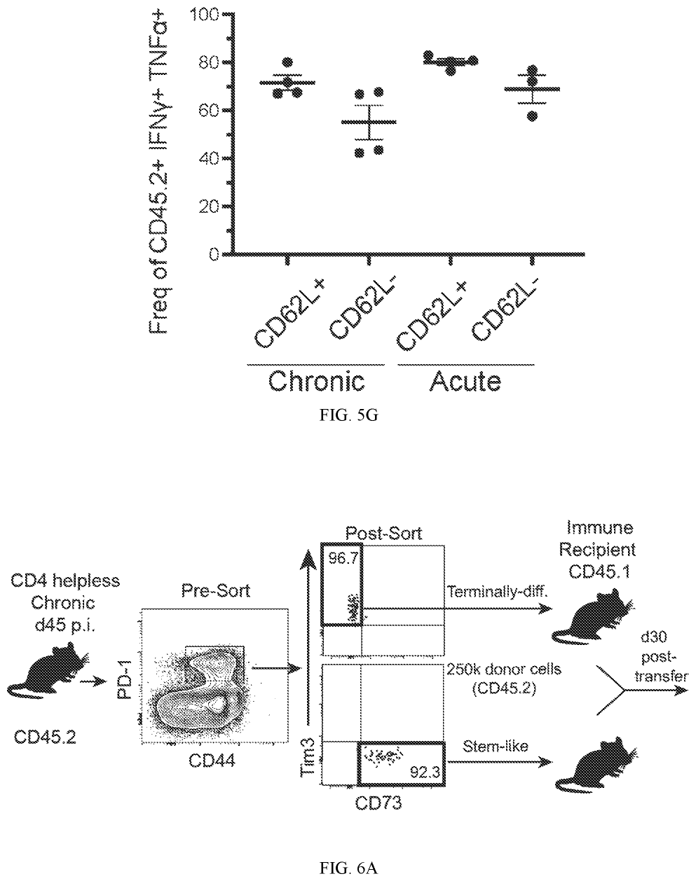

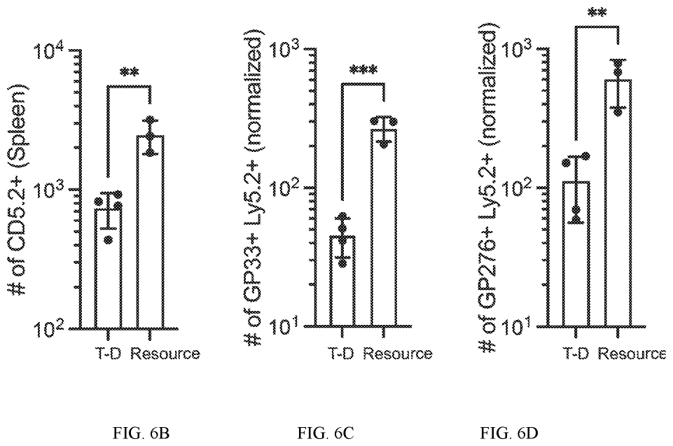

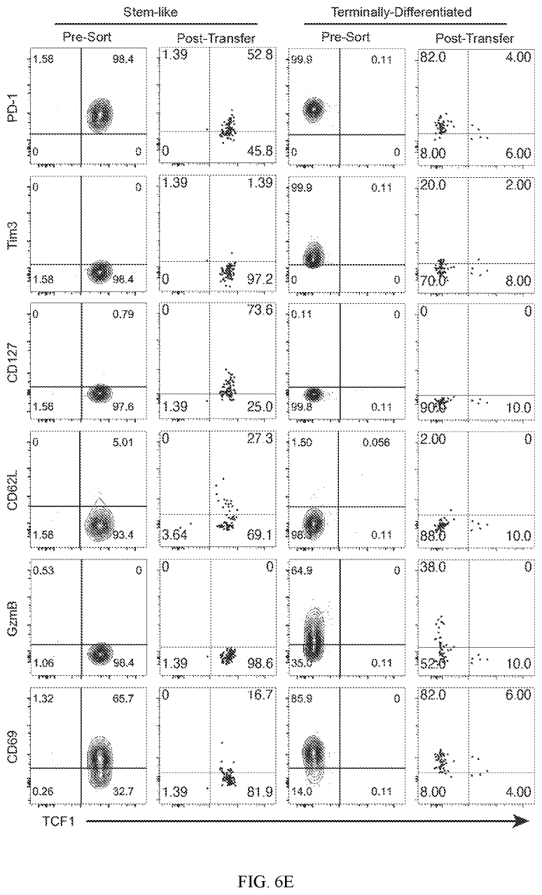

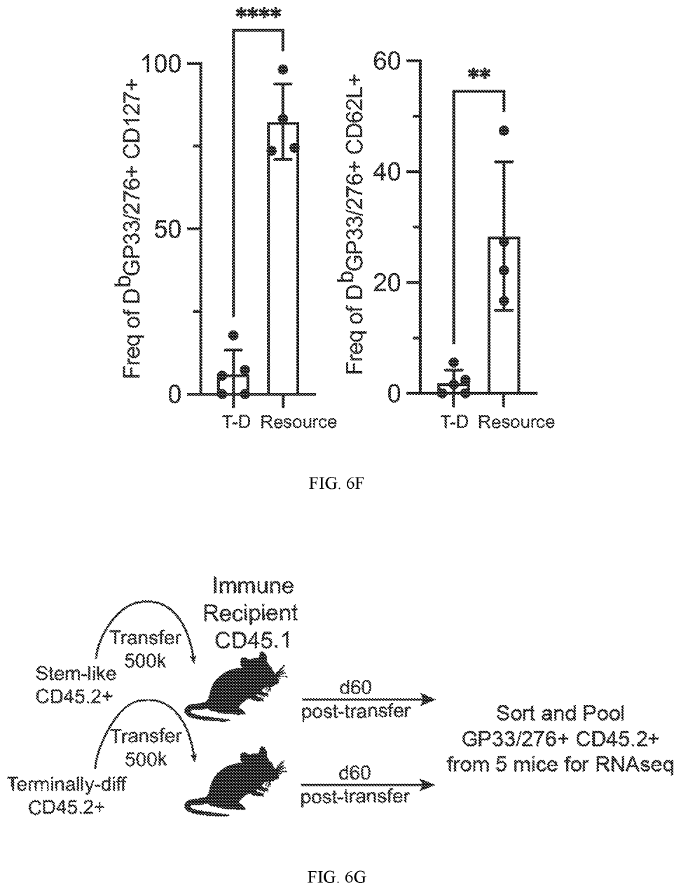

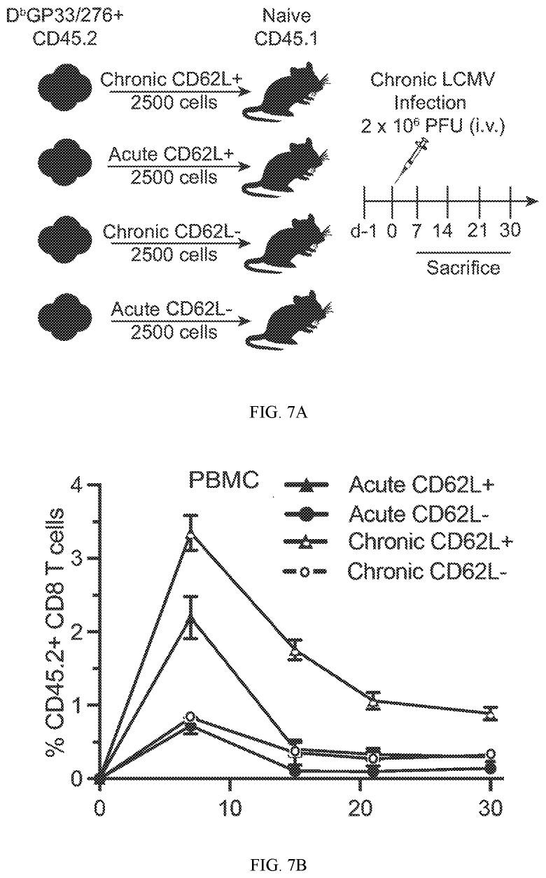

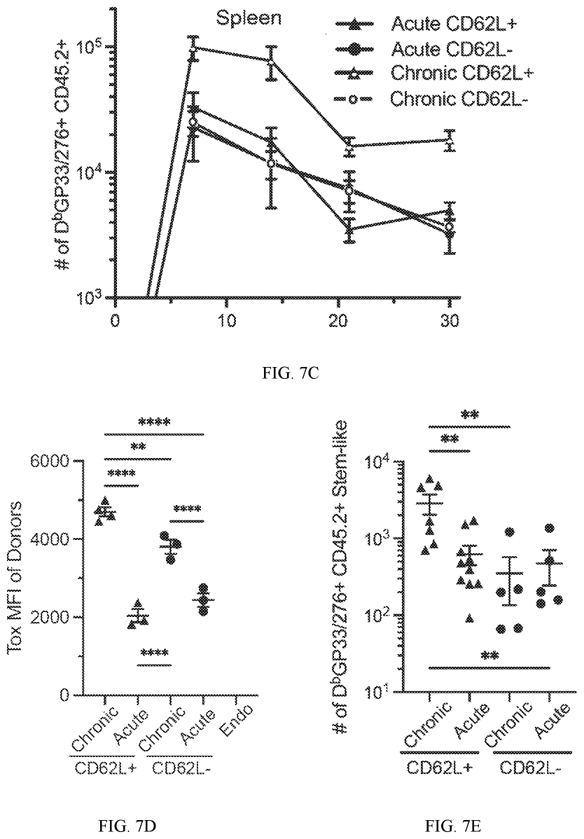

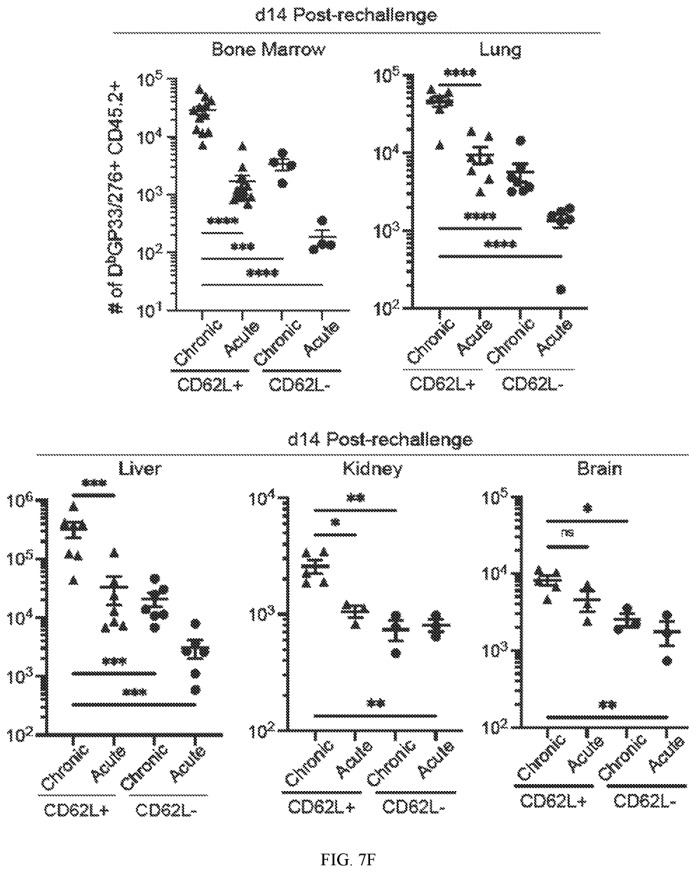

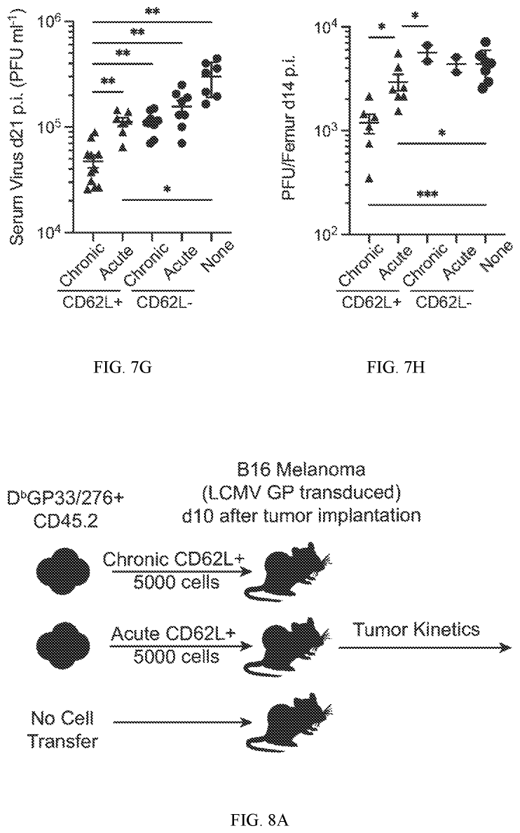

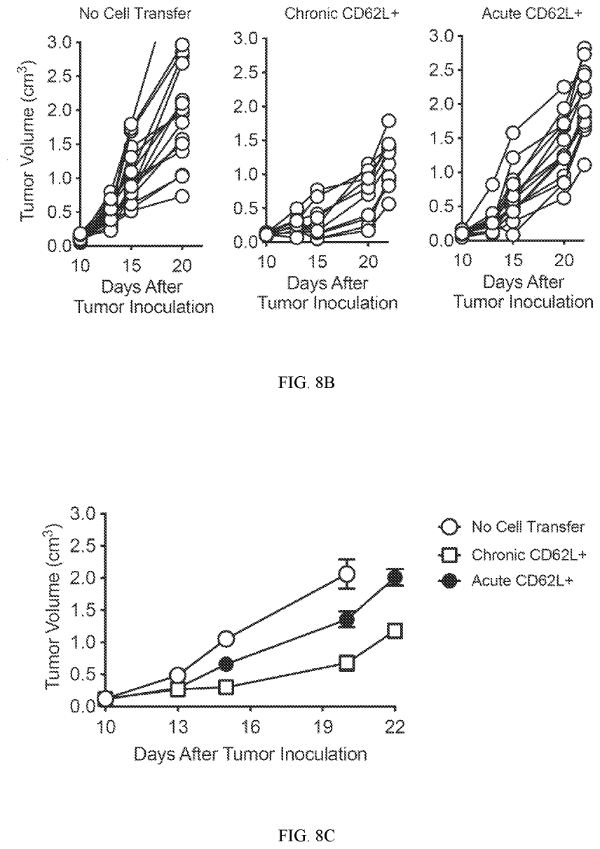

To investigate the functional differences of acute and chronic memory subsets, these LCMVspecific subsets were sorted as previously mentioned and equal numbers of donors were transferred to congenically marked naïve recipients. The recipient mice were infected with acute LCMV one day post-transfer (FIG. 5A). Kinetics in the PBMC showed that the CD62L+ subsets of acute and chronic memory cells had better recall. The CD62L− chronic memory subset had the lowest recall in the blood after rechallenge (FIG. 5B). In the spleen after 40 days p.i., the two CD62L+ acute and chronic memory cells had similar frequencies and numbers which were higher than the two CD62L− acute and chronic memory cells (FIG. 5C).

Homeostatic proliferation (TCF1+Ki67+) of the donor cells was assessed. Interestingly the two CD62L+ subsets had the highest number of cells undergoing homeostatic proliferation but the CD62L+ chronic memory had statistically significant increase in the number of cycling cells (FIG. 5D).

Since TOX and PD-1 were upregulated in the chronic memory subset after Cl-13 clearance, expression of these molecules were investigated. TOX and PD-1 expression were the highest in the CD62L+ and CD62L− chronic memory subsets compared to any other subsets even after an acute infection suggestion that the important marks of enduring chronic stimulation are maintained (FIGS. 5E and 5F). Seems that these enduring characteristics are hallmark of chronic memory cells that contribute to their unique phenotype and function. They have the ability to produce the cytokine. Sequential loss of cytokine production is characteristic of T cell exhaustion. All subsets except the CD62L− chronic memory subset had significant frequencies of IFNγ and TNFα co-expressing donors (FIG. 5G). These result suggest that the CD62L+ chronic memory subset has similar functional capabilities to the CD62L+ acute memory subset which are thought to be the epitome of memory cells. This is particularly interesting because the chronic memory cells expressed higher levels of TOX and PD-1. Perhaps these molecules are not too bad after all and must be important of the imprinting and survival of cells during chronic antigenic stimulation. Further, seeing that the CD62L− chronic memory donor cells performed the least optimally in both the numbers and function after recall corroborates that this subset is most likely remnants of terminally-differentiated cells found during chronic LCMV infection.

### Adoptive Cell Therapy (ACT)

Adoptive cell therapy (ACT) utilizes autologous T cells that can be expanded and engineered to recognize target cells (such as cancer) which can lead to disease regression. However, limitation in the persistence of these adoptively transferred T cells, particularly CD8 T cells, have hindered efficacy. The stem-like CD8 T cells that sustain the response during the chronic viral infection persist and differentiate into the stem-like chronic memory cells which have adapted to survive long-term after antigen clearance. These cells can be isolated and used for adoptive cell therapies for cancer, chronic viral infection, and/or other chronic diseases with better efficacy due to their superior recall potential and persistence. Furthermore, the adoptively transfer of these cells can be paired with PD-1 blockade agents which can significantly bolster the effector differentiation of these transferred cells.

Isolating and utilizing chronic memory CD8 T cells would be used to solve the issues of persistence and functional impairments of ACT in the context of cancer, both hematologic and solid tumors, and chronic viral infections. This provides superior durability and functional potential to control chronic viral infections and cancer. In vivo murine model of chronic viral infection have shown that the stem-like CD8 T cells that sustain the response during the chronic viral infection persist and differentiate into the stem-like chronic memory cells which have adapted to survive long-term after antigen clearance. These cells have the best ability to survive and differentiate into effector CD8 T cells upon rechallenge in hostile highly inflammatory settings compared to acute memory cells that are currently being utilized for ACT. Therapeutically, the group of mice that received the transfer of chronic memory cells had the greatest reduction of chronic viral burdens. This superior reduction in the viral burden would be translated to murine tumor models. The potential of chronic memory cells to best persist and quickly differentiate into functional effector cells are desirable for improving current ACT limitations. These cells can be isolated and used for adoptive cell therapies for cancer, chronic viral infection, and/or other chronic diseases with better efficacy due to their superior recall potential and persistence. Furthermore, the adoptively transfer of these cells can be paired with PD-1 blockade agents which can significantly bolster the effector differentiation of these transferred cells. In addtion, transcriptionally and epigenetically reprogram cells to resemble the chronic memory cells for use in cellular therapies are contemplated,

Transcriptional genes and epigenetic genes contemplated include differentially methylated promoter sites between acute and chronic CD62L+ memory cells such as the Plac8 gene at region −1195, Itpr2 gene at region 2846, Cd244a gene at region -8432, Hdac7 gene at region −11490, Nr4a2 gene at region −2775, Ccr7 gene at region 13027, Brd4 gene at region 180, Zeb2 gene at region −4166, Foxn2 gene at region 13759, Ly6e gene at region 2204, Axl gene at region −6090, Il2ra gene at region 1516, Smad4 gene at region 4172, Il1rl2 gene at region 24843, Csfl gene at region 9399, Slamf6 gene at region 1971, Runx3 gene at region 6855, Cd9 gene at region −35579, Foxo3 gene at region −6211, Ikzf4 gene at region 6837, Ccl4 gene at region −4800, Tigit gene at region −4856, Gata6 gene at region −27453, Eomes gene at region 1624, Kif2b gene at region −330910, Irf2 gene at region 23960, Bcl2115 gene at region −4260, Cd200r2 gene at region 3780, Cxcl10 gene at region −635, Batf gene at region 1381, Cdh6 gene at region 29290, Pdcd1 gene at region 3097, Nek7 gene at region −4016, Sox3 gene at region −35776, and/or Tox gene at region −125027.

In cetain embodiments, genes are up/downregulated with unique epigenetic signatures in T cells isolated from patients and manipulated transcriptionally and/or epigenetically to resemble the cells disclosed herein, e.g., stem-like chronic memory cells.

In certain embodiments, this discloure relates to epigenetic composition of these cells and its dependents similar to the transcriptional composition.

In certain embodiments, this discloure relates to modification/induction of the following genes TOX, Satb 1 (special AT-rich sequence binding protein 1), Maf (proto-oncogene c-Maf), and Eomes (Eomesodermin) in a cell to produced cells disclosed herein, e.g., stem-like chronic memory cells or stem-like chronic resource cells or other cell expression or marker profiles as disclosed herein. In certain embodiments, expression may be induced by exposure of cells to a vector(s) encoding the gene in operable combination with a promoter (heterologous) or by inserting into cells DNA or RNA, e.g., mRNA encoding the gene(s). Vector or other nucleic acids encoding the genes may be individual or combined into one or more constructs separated by self-cleaving preptides, mulitple promoters, and/or an internal ribosome entry site (IRES).

TABLE 1
Differentially expressed genes between acute
and chronic CD62L+ memory CD8 T cells

| Gene | baseMean | log2FoldChange | lfcSE |
|---|---|---|---|
| Trgv2 | 1317.4788 | 6.83964809 | 0.2792584 |
| Serpina3g | 3367.08376 | 2.41674507 | 0.11419997 |
| Klre1 | 1278.88609 | 4.43607517 | 0.26631932 |
| Trgc2 | 3686.16035 | 6.31786361 | 0.38613718 |
| Cd38 | 930.63623 | 2.03193404 | 0.16744427 |
| Anxa2 | 4844.75413 | 1.17682834 | 0.10423522 |
| Klrc1 | 5936.094 | 1.03313561 | 0.09379216 |
| Trgc4 | 920.602497 | 11.3659381 | 1.03845286 |
| Trgv1 | 153.489927 | 7.27253493 | 0.71827501 |
| Eomes | 2188.32976 | 1.26970278 | 0.13037086 |
| Pdcd1 | 5284.44991 | 2.70642045 | 0.27841358 |
| Tox | 2522.63022 | 2.99533627 | 0.32618228 |
| Ldhb | 300.239543 | 2.92858225 | 0.32276412 |
| Klrg1 | 1530.60244 | −1.3782831 | 0.15321995 |
| Prr5l | 679.080557 | 5.48555731 | 0.61102935 |
| Maf | 240.086717 | 4.08379944 | 0.45561444 |
| Gm2a | 1534.83775 | −1.2045145 | 0.13575346 |
| Bex3 | 948.009929 | 1.28472168 | 0.14624571 |
| Acot7 | 2902.3613 | 1.00017508 | 0.1145053 |
| Dgkh | 460.110963 | −1.9587255 | 0.2243346 |
| Lmna | 924.61507 | 2.19692879 | 0.25180294 |
| Plscr4 | 157.921162 | 4.11464702 | 0.47621358 |
| Serpina3f | 326.916641 | 2.90702439 | 0.33686907 |
| Gzmk | 3889.87931 | 1.43427023 | 0.17021263 |
| Tceal9 | 416.124264 | 1.3427128 | 0.16278748 |
| Myadm | 711.307277 | 2.24404233 | 0.27537149 |
| Fgl2 | 1064.30598 | 1.56706031 | 0.19342198 |
| Rnf130 | 736.966581 | 1.16786758 | 0.14411715 |
| Cpne7 | 469.692503 | 2.16488008 | 0.26805443 |
| Ogfrl1 | 108.862546 | 2.8679675 | 0.35583413 |
| Satb1 | 2712.46314 | −1.0283695 | 0.12852428 |
| Wfikkn2 | 43.8669243 | −7.1813501 | 0.90525691 |
| Nr3c2 | 100.727574 | 4.05101142 | 0.51582623 |
| Casp1 | 1433.90376 | 1.17602235 | 0.14991352 |
| Osbpl3 | 2015.1708 | 1.50437851 | 0.19234941 |
| Aplp1 | 630.047551 | 1.82791922 | 0.23405882 |
| Ikzf3 | 4537.73397 | 1.05543724 | 0.13614114 |
| Vmp1 | 4013.35502 | 1.05634257 | 0.13926971 |
| Fcgr2b | 682.352697 | −2.058989 | 0.271883 |
| Nkg7 | 55263.4441 | 0.73305581 | 0.09785789 |
| Slc2a3 | 1373.76003 | −0.8730061 | 0.1170074 |
| Tnfsf13b | 160.665346 | 2.49006387 | 0.33586877 |
| Lgals1 | 11921.2592 | 0.61994411 | 0.08516071 |
| Iigp1 | 164.349401 | 2.60547759 | 0.36081179 |
| Gimap7 | 5560.05022 | 0.79138608 | 0.11072739 |
| Ptpn11 | 1335.38099 | 1.02460558 | 0.14350406 |
| Cd8b1 | 22978.6324 | −0.6853679 | 0.09945805 |
| Lgals3 | 3424.86301 | 1.94610169 | 0.28262316 |
| B4galt4 | 186.39204 | 3.25615531 | 0.48252502 |
| Ms4a4a | 210.360199 | 2.48526185 | 0.36892358 |
| Zbtb32 | 497.714732 | 2.35538694 | 0.35376645 |
| BC064078 | 129.648406 | 2.17642158 | 0.3280798 |
| Sesn1 | 780.2795 | −0.9800937 | 0.14770137 |
| Plscr1 | 586.115537 | 1.2563237 | 0.19098873 |
| Kcnip3 | 214.252333 | 2.08182771 | 0.31702746 |
| Armc7 | 4534.61909 | 0.8721527 | 0.13289039 |
| Ssh1 | 633.961846 | 0.90969155 | 0.13871143 |
| Cish | 771.243254 | 1.578352 | 0.24210156 |
| Tacc1 | 1741.31798 | −0.5098909 | 0.07831812 |
| Slc27a4 | 642.741984 | 1.31284618 | 0.20282586 |
| Pvrig | 226.657801 | 2.18448167 | 0.33861718 |
| Litaf | 667.810449 | 1.44389753 | 0.2261267 |
| Tmem154 | 982.020723 | −0.9015883 | 0.14198215 |
| 2310001H17Rik | 1088.78707 | 0.82974306 | 0.13094381 |
| Ildr1 | 809.007964 | 1.01174379 | 0.16136909 |
| Ociad2 | 174.308357 | 4.24477192 | 0.67941335 |
| Cyp4f16 | 252.343203 | 1.49443834 | 0.23989569 |
| Acadl | 1687.48659 | 0.62398145 | 0.10048512 |
| Pygl | 218.542426 | 1.83858082 | 0.29794812 |
| Gcnt1 | 87.3774417 | 3.42278046 | 0.5559358 |
| Ttyh3 | 277.219922 | −1.4579357 | 0.23776149 |
| Metrnl | 328.614881 | 1.66789926 | 0.27273922 |
| Lpin1 | 2701.86014 | 0.83818875 | 0.13719293 |
| Lratd1 | 118.039553 | 9.11059911 | 1.49321805 |
| Pros1 | 336.010832 | 1.86464243 | 0.30864519 |
| Vim | 5378.85144 | 0.65910029 | 0.1096548 |
| Hic1 | 289.32015 | 2.18233945 | 0.36546902 |
| Klf10 | 1224.53007 | 0.73408178 | 0.12306097 |
| Tigit | 1130.3912 | 2.32424221 | 0.38989875 |
| Cldnd1 | 1263.1147 | 0.81978937 | 0.13845848 |
| Furin | 645.654984 | −1.0181661 | 0.17227155 |
| Qpct | 464.804202 | 1.77906078 | 0.30314517 |
| Tle1 | 191.597976 | −2.3327167 | 0.39919136 |
| Srebf2 | 1267.11475 | −0.6886636 | 0.11799118 |
| Nfatc3 | 2678.47079 | −0.6982001 | 0.11964617 |
| Itgb2 | 20320.0654 | 0.44004543 | 0.07555532 |
| Rnf128 | 190.660912 | 1.86423009 | 0.32046094 |
| Asb2 | 251.564873 | 1.86581123 | 0.32182225 |
| Vwa5a | 825.937061 | 1.06448229 | 0.18366835 |
| Tppp3 | 106.646969 | 3.61810462 | 0.62507635 |
| Tnfrsf8 | 96.6237905 | 4.46958563 | 0.7748532 |
| Ica1 | 84.2847722 | 2.98921999 | 0.51950124 |
| Klrk1 | 9229.94962 | 0.50223933 | 0.08760858 |
| Abcg2 | 188.320165 | 1.79841705 | 0.31402464 |
| Tmbim4 | 865.233745 | −0.7564028 | 0.13324899 |
| Stx11 | 827.238293 | 0.86404253 | 0.15302369 |
| Trim2 | 74.6863047 | 2.38835084 | 0.4237911 |
| Cyth3 | 350.073693 | 2.04664532 | 0.36387677 |
| Ybx3 | 1588.54576 | 0.57235658 | 0.10216901 |
| Hif1a | 1221.68491 | −0.8996972 | 0.16132984 |
| Cmtm6 | 3652.68109 | −0.4954025 | 0.08918441 |
| Nr4a2 | 1040.7754 | 3.42154768 | 0.61940805 |
| Il7 | 60.988367 | 2.13157593 | 0.38590973 |
| Dtx4 | 268.931806 | 1.39735075 | 0.25330796 |
| Prr13 | 6042.20893 | 0.73299935 | 0.13302138 |
| Itgax | 774.840154 | −1.3183076 | 0.24018065 |
| Pctp | 238.299257 | 1.09832269 | 0.20122424 |
| Bhlhe40 | 2108.89523 | 1.04933365 | 0.19264684 |
| Rasgef1b | 276.55471 | 2.32247679 | 0.42753722 |
| Cxcr5 | 809.131326 | 2.82651984 | 0.5216489 |
| Sh2d1a | 2284.95182 | 0.70441994 | 0.13031981 |
| Akr1e1 | 188.479796 | 1.74664217 | 0.32341643 |
| Tmem51 | 151.334592 | 2.72223008 | 0.50460091 |
| Pde3b | 1102.88454 | −0.9634036 | 0.17851624 |
| Tanc1 | 263.723663 | −1.6329197 | 0.30263948 |
| Tbc1d2 | 31.7339556 | −7.4372616 | 1.37936227 |
| Tspan3 | 1389.91223 | 0.96638031 | 0.17991219 |
| Septin4 | 1125.15906 | 1.40943433 | 0.26401044 |
| Sidt1 | 3689.0742 | −0.6259646 | 0.1175008 |
| Rasl12 | 220.403584 | 1.46358242 | 0.27625666 |
| Map3k3 | 1681.68118 | −0.6238639 | 0.11776243 |
| Lrrk1 | 1456.90922 | 0.70423165 | 0.13311809 |
| Trbv19 | 486.742442 | −5.063675 | 0.95724084 |
| Chst12 | 1081.67642 | 0.85192843 | 0.16112105 |
| Naip5 | 167.641596 | 2.1887204 | 0.41726808 |
| S100a6 | 12269.3426 | 0.54742108 | 0.10447978 |
| Ppp1r11 | 1434.29797 | 0.48119223 | 0.09232958 |
| Sytl1 | 278.653966 | −1.1866603 | 0.22799618 |
| Dact2 | 23.4349211 | 7.44364795 | 1.43377584 |
| Stk39 | 868.731671 | 0.7108999 | 0.13695404 |
| Nsmaf | 5561.18724 | 0.46983237 | 0.09052725 |
| Gnb4 | 113.098241 | 2.05947822 | 0.39716502 |
| Wls | 1032.1982 | 1.14184589 | 0.22041184 |
| Fcgrt | 726.883114 | −1.2350473 | 0.23861818 |
| Cmah | 1194.36945 | −0.8834338 | 0.17169642 |
| S100a11 | 3434.4616 | 0.57327015 | 0.11183166 |
| Dleu2 | 109.97622 | −1.9180538 | 0.37467484 |
| Gm35363 | 83.0320092 | 6.90869531 | 1.35204805 |
| Stim1 | 2008.42339 | −0.4894105 | 0.09606098 |
| Fam241a | 221.692819 | −1.3996716 | 0.27484459 |
| Atp6v0d2 | 452.711722 | 2.81991058 | 0.55438197 |
| Gm11454 | 103.13499 | 5.90054889 | 1.1632309 |
| Epha3 | 279.394996 | 3.22522282 | 0.63564088 |
| Efhd2 | 8473.86865 | 0.53927286 | 0.1063362 |
| Arsb | 688.450827 | 0.92721079 | 0.18310726 |
| Kif5c | 193.94492 | 1.902887 | 0.37642748 |
| Cpm | 336.554062 | −1.7708985 | 0.35157867 |
| Sulf2 | 35.1814765 | 6.59477573 | 1.31677661 |
| Kcnn4 | 4908.74263 | 0.54406081 | 0.10869296 |
| Tmem171 | 133.217268 | 1.53724658 | 0.30736541 |
| Peli1 | 3325.48694 | −0.4797428 | 0.09639045 |
| Pdgfb | 361.162024 | 1.37503574 | 0.27681426 |
| App | 224.7931 | 1.95287556 | 0.39338982 |
| Rnf19b | 806.303416 | 0.81878325 | 0.1652512 |
| Hspa2 | 37.6243346 | 3.70732498 | 0.74858703 |
| Mrtfa | 1619.93261 | −0.5485856 | 0.11115094 |
| Racgap1 | 2442.78664 | 0.64237444 | 0.13061984 |
| Ryr1 | 120.129906 | 2.81415527 | 0.572947 |
| Gem | 468.672666 | 1.57080763 | 0.31976829 |
| Cd72 | 1578.95573 | 0.69224126 | 0.14096156 |
| Cenpj | 198.713791 | −1.4476748 | 0.2948055 |
| Rbm47 | 96.9281633 | −2.976903 | 0.60601068 |
| Cd40lg | 60.182332 | −3.2448158 | 0.66384532 |
| Itgb3 | 58.7554171 | −4.3632435 | 0.89296885 |
| Gzmb | 3995.79914 | −1.3192575 | 0.27018601 |
| Atp11b | 2728.26539 | −0.5599306 | 0.11491846 |
| Dop1a | 257.996489 | −1.4435343 | 0.29641851 |
| Btg2 | 3623.4787 | −0.4816119 | 0.09892252 |
| Rapgef6 | 2258.49914 | −0.5479689 | 0.11283792 |
| Cela1 | 316.557782 | 1.92649009 | 0.39708521 |
| Ywhah | 2568.666 | 0.3705086 | 0.07640779 |
| Plac8 | 752.507573 | −2.1213479 | 0.43773817 |
| Cd200r4 | 800.971009 | 0.79972564 | 0.16536175 |
| Ube2l6 | 288.865759 | 1.7374641 | 0.36007535 |
| Foxn3 | 1770.72141 | −0.484637 | 0.10046466 |
| Napsa | 278.288675 | 1.97409131 | 0.40974697 |
| Klrb1c | 1149.22146 | −1.6825984 | 0.34928923 |
| Atp1a1 | 2062.46374 | −1.3556231 | 0.28184367 |
| Ppp2r2c | 38.0252482 | 3.35152272 | 0.69808696 |
| Pls3 | 73.4711305 | 2.94505272 | 0.61482663 |
| Rasgef1a | 54.3783843 | 3.07277347 | 0.64191469 |
| Zc2hc1a | 89.0467322 | 2.22454952 | 0.46489878 |
| Sqle | 173.669435 | −1.383541 | 0.28949845 |
| Ptpn6 | 4296.57812 | −0.5703482 | 0.11958165 |
| Il2rb | 18539.9602 | 0.42173758 | 0.08886011 |
| Srgap2 | 550.82854 | −0.9426285 | 0.19868668 |
| Gm44175 | 248.774209 | −1.1838194 | 0.24970849 |
| Samd3 | 1750.72284 | 0.71314876 | 0.15104697 |
| Mcub | 302.708906 | 1.32681259 | 0.28195214 |
| Adrb1 | 164.247764 | 1.76416397 | 0.37594579 |
| Stk38 | 3663.73206 | −0.4204796 | 0.08981071 |
| Ncf4 | 1593.09399 | 0.64733955 | 0.13897614 |
| Zcchc18 | 905.201992 | 0.72056913 | 0.15503389 |
| Gas7 | 340.576837 | 1.05190244 | 0.2265997 |
| Ttr | 363.35387 | 1.88555283 | 0.40783622 |
| Xylt1 | 263.76378 | −1.3954079 | 0.30206923 |
| Il12rb2 | 208.54933 | −1.8957844 | 0.41033923 |
| Pde4b | 816.468583 | −0.8316094 | 0.18009855 |
| Syne1 | 420.980118 | −1.1389759 | 0.2466847 |
| Sh3yl1 | 63.7699712 | 2.36567635 | 0.51288669 |
| H2-Q5 | 1929.63503 | 0.57925435 | 0.12562414 |
| Rictor | 1955.4806 | −0.6580257 | 0.14267916 |
| Oas1a | 643.474561 | 0.71477572 | 0.15512229 |
| Scly | 604.998058 | 0.69513527 | 0.15130009 |
| Tmem159 | 193.430805 | 1.02936876 | 0.22413257 |
| Galm | 377.867538 | 1.19002252 | 0.25919031 |
| Ly6a2 | 94.6517624 | −6.1444351 | 1.34023444 |
| Dapk2 | 584.072125 | 1.07713592 | 0.23509077 |
| Inpp5f | 220.332483 | −1.1798272 | 0.25756596 |
| B630019K06Rik | 42.1399846 | 2.34624464 | 0.51344224 |
| Rin3 | 1180.30096 | −0.711207 | 0.15602003 |
| Stat5b | 1911.78314 | −0.6048426 | 0.13297659 |
| Ncoa3 | 594.934824 | −0.8170068 | 0.17985072 |
| Fbxw11 | 954.499507 | −0.7478111 | 0.16548223 |
| Crip2 | 253.243254 | 1.55884902 | 0.34523279 |
| Pacs2 | 419.476131 | −0.8957037 | 0.19840221 |
| H2-Q7 | 21070.5537 | 0.3101032 | 0.06875653 |
| Ahcy | 249.821103 | −1.0090883 | 0.22399379 |
| Agrn | 35.0040804 | −7.2441905 | 1.61098803 |
| Pkp4 | 221.732938 | −2.0582258 | 0.45851402 |
| Galnt10 | 1943.0741 | −0.7448861 | 0.16615196 |
| Ctsd | 14238.2438 | 0.28917072 | 0.06467021 |
| Tpd52 | 339.109435 | 0.93080398 | 0.20847594 |
| Setbp1 | 152.419361 | 2.60675185 | 0.58425598 |
| Tbl1xr1 | 669.336128 | −0.6368208 | 0.14277122 |
| Cyp2s1 | 53.298704 | −2.6203859 | 0.58776572 |
| Zfp512 | 1140.75581 | 0.61657565 | 0.13837706 |
| Bicd1 | 114.707068 | 2.20134282 | 0.49455375 |
| Sipa1l1 | 539.789624 | −0.9504417 | 0.21348493 |
| Ccnd2 | 8517.84788 | 0.40501791 | 0.09114705 |
| Ywhaq | 4317.45915 | 0.39139796 | 0.08816487 |
| Fam174b | 116.001975 | 2.73612248 | 0.61808786 |
| Cdk4 | 3535.57874 | 0.33995675 | 0.07681223 |
| Xcl1 | 832.672311 | 2.34791739 | 0.53118388 |
| Osr2 | 56.5348457 | 3.72906746 | 0.84444031 |
| Crlf3 | 3151.89562 | −0.4615324 | 0.10453567 |
| Jpt1 | 6789.77438 | 0.44265858 | 0.10084742 |
| Car5b | 336.218832 | 1.42294969 | 0.32457994 |
| Cers4 | 1256.85844 | 0.71546921 | 0.16320177 |
| Rapgef2 | 357.423469 | −1.3100188 | 0.2996428 |
| Slamf1 | 436.661639 | −2.4075244 | 0.55082233 |
| Pdk1 | 945.653214 | −0.7723552 | 0.17682149 |
| Mapk12 | 94.3215872 | 1.55174225 | 0.35660554 |
| Gm14125 | 124.065281 | 1.76732494 | 0.40639874 |
| Frmd6 | 118.762483 | −2.2959705 | 0.5283098 |
| Prmt2 | 523.579768 | 0.76131843 | 0.17530849 |
| P2ry12 | 35.2668721 | −4.8781738 | 1.125237 |
| Myo1g | 5000.14473 | 0.47751002 | 0.11037753 |
| Ighm | 3378.15368 | −0.7168931 | 0.16579175 |
| Slc25a24 | 820.33142 | 0.64794969 | 0.15017209 |
| Cd22 | 570.761566 | 2.46837864 | 0.57233459 |
| Scamp3 | 1991.52505 | 0.45315159 | 0.10527048 |
| Psen2 | 897.504833 | 0.89316928 | 0.20762559 |
| Cd86 | 390.348066 | 0.93673148 | 0.21792719 |
| Tnfsf4 | 205.736026 | 3.36029929 | 0.7820916 |
| Cd82 | 12978.3373 | 0.43000244 | 0.10038711 |
| Arf6 | 4572.81386 | 0.42016191 | 0.0981955 |
| Smg6 | 630.428441 | −0.7566903 | 0.1769344 |
| Cd9 | 592.992796 | −0.8227021 | 0.19240583 |
| Fyn | 11314.8054 | 0.35711263 | 0.08353936 |
| Cd160 | 2075.31239 | 0.79028155 | 0.18492615 |
| AU020206 | 2164.48339 | 0.69327726 | 0.16236327 |
| Cdc42se2 | 3627.00079 | −0.3109394 | 0.07285656 |
| Lilrb4b | 941.592242 | 0.97708042 | 0.22918902 |
| Tnfrsf13c | 246.946246 | 1.12436275 | 0.26406778 |
| 6330403K07Rik | 182.654248 | 1.32486033 | 0.31129616 |
| Emp3 | 5254.09226 | 0.38743801 | 0.09118998 |
| Dtx1 | 4423.45823 | −0.6491393 | 0.15295378 |
| Ctdsp2 | 856.457813 | −0.5971111 | 0.14129594 |
| Nr4a1 | 194.465324 | 1.5528096 | 0.36830499 |
| Stard3nl | 908.972566 | 0.69692686 | 0.16531126 |
| Cpq | 79.1357227 | 3.32919985 | 0.78989393 |
| Iglc2 | 307.331773 | 1.34278547 | 0.31924932 |
| C1qtnf6 | 59.4218636 | 2.5535327 | 0.6079125 |
| Atg3 | 1550.05971 | 0.36983555 | 0.08838088 |
| Ephb6 | 513.542962 | 1.29924421 | 0.31102982 |
| Hlcs | 378.141124 | −0.7990678 | 0.19206462 |
| Cit | 229.474535 | 1.19756722 | 0.2891782 |
| Ran | 3441.41779 | 0.32269717 | 0.07793101 |
| 1700017B05Rik | 382.082683 | −1.073041 | 0.25935266 |
| Pwwp2b | 69.2001281 | 2.27086125 | 0.54898298 |
| Alox8 | 216.503203 | 1.15955334 | 0.28056197 |
| Bbs9 | 292.520456 | −1.1331809 | 0.27438118 |
| Pak6 | 94.3811989 | −1.7998356 | 0.43588455 |
| Ctss | 2077.2613 | 0.50257914 | 0.12176193 |
| Txn1 | 2159.2426 | 0.44023508 | 0.10676536 |
| Tob1 | 549.781985 | −0.9885297 | 0.24023409 |
| Lamc1 | 112.490669 | −2.6932129 | 0.65452987 |
| Tanc2 | 64.4317125 | −2.5371423 | 0.61804133 |
| Prss2 | 194.493436 | 11.5771316 | 2.82177488 |
| Nin | 478.094524 | −0.8557471 | 0.20861251 |
| Itgam | 27.6826489 | −4.9587498 | 1.2097158 |
| Batf | 2188.21079 | 0.43579181 | 0.10651061 |
| Pqlc3 | 990.800919 | 0.71437089 | 0.17499512 |
| Cd79b | 666.889436 | −1.8982172 | 0.46555177 |
| H2az1 | 4942.30306 | 0.33781971 | 0.0828958 |
| Ankrd13a | 2523.2866 | −0.50203 | 0.12321983 |
| Phf3 | 724.477671 | −0.5865902 | 0.14414164 |
| Cflar | 770.599344 | −0.7350755 | 0.18098907 |
| Tmem131l | 2238.25316 | −0.4276841 | 0.10543617 |
| Mlec | 1028.28028 | −0.7582593 | 0.18712916 |
| Il10ra | 3477.49893 | 0.48646352 | 0.1200811 |
| Gm15228 | 21.600427 | 3.65408503 | 0.90259562 |
| Cnot6l | 1668.83731 | −0.6113849 | 0.15132668 |
| Crip1 | 10569.0762 | 0.42810235 | 0.10626288 |
| Plod2 | 83.5067182 | 2.73162448 | 0.68043874 |
| Tppp | 53.6197976 | 2.15843825 | 0.53788727 |
| Itgad | 196.023496 | 1.95181794 | 0.48671086 |
| Cd200r1 | 819.530967 | 1.10295168 | 0.27572165 |
| H2-Ob | 486.710555 | −1.1820603 | 0.29611239 |
| Rnf165 | 38.3826386 | 5.47803975 | 1.37343722 |
| Ucp2 | 11867.4916 | 0.36906693 | 0.09256563 |
| Dpp4 | 1978.97393 | −0.5462832 | 0.13700641 |
| Jak3 | 2638.99043 | 0.64968766 | 0.16300797 |
| Nab2 | 153.609561 | −1.1828118 | 0.29700242 |
| Chd4 | 961.449652 | −0.6314441 | 0.1588455 |
| Xbp1 | 1740.87339 | 0.47935914 | 0.12070732 |
| Trbc1 | 10061.0386 | 0.53655302 | 0.13530919 |
| Wtap | 574.111619 | 0.50850524 | 0.12830138 |
| Rnaseh2c | 1212.60174 | 0.55872118 | 0.14100284 |
| Zeb2 | 758.605763 | −1.1130575 | 0.28120398 |
| Reep5 | 1699.37777 | 0.50771417 | 0.12878205 |
| Tbc1d1 | 707.004606 | −0.6495763 | 0.16479752 |
| Pdia6 | 2418.29821 | 0.43845071 | 0.11127525 |
| Orai2 | 2015.01513 | −0.4686754 | 0.11944664 |
| Gm8817 | 183.868494 | 0.98057908 | 0.25031933 |
| Lrrc8c | 2440.48206 | 0.40752522 | 0.10404105 |
| Chst2 | 326.389415 | 7.58766511 | 1.93803898 |
| Serp2 | 184.318079 | 1.04538433 | 0.26727463 |
| Exosc8 | 1639.60001 | 0.48194087 | 0.1232401 |
| Bex2 | 47.453333 | 1.81625785 | 0.46477495 |
| Gm28053 | 102.431281 | 1.27477942 | 0.32646812 |
| Ranbp10 | 712.369308 | 0.5486002 | 0.14051591 |
| Cyp17a1 | 44.9878671 | 2.81111795 | 0.7202398 |
| Edem1 | 1727.42557 | −0.4662276 | 0.11989896 |
| S1pr5 | 2494.99766 | −1.0849736 | 0.28017102 |
| Ipcef1 | 967.936639 | −0.5770033 | 0.14929402 |
| Prkcb | 2403.3742 | −0.533804 | 0.13821007 |
| Bcl9l | 283.877714 | −0.7883988 | 0.20422018 |
| Ylpm1 | 352.777453 | −0.8821181 | 0.22866535 |
| Zfp646 | 583.311478 | −0.9740501 | 0.25259287 |
| Agpat4 | 500.974476 | −0.8438873 | 0.21951413 |
| Pafah1b3 | 180.122773 | 0.8752988 | 0.22787491 |
| Psmb8 | 8825.79397 | 0.38805411 | 0.10106993 |
| Hgfac | 42.5737433 | 2.73500779 | 0.71271379 |
| Spc25 | 47.0539027 | 2.30839928 | 0.60196842 |
| Selenow | 2966.18081 | 0.43086194 | 0.11246534 |
| Gm4208 | 53.4069601 | −2.9426896 | 0.76808395 |
| Ap3m2 | 640.723693 | −0.789822 | 0.20670628 |
| Enpp5 | 266.380017 | 0.94535266 | 0.24830014 |
| Frmd4a | 146.210688 | 1.26343415 | 0.33207645 |
| Smap1 | 783.629125 | 0.61698235 | 0.16271519 |
| Gpr18 | 2857.67611 | 0.55325563 | 0.14588785 |
| H2-Q6 | 15574.785 | 0.31456461 | 0.08300951 |
| Calm1 | 18544.5555 | 0.28475074 | 0.075138 |
| Fam168b | 1810.69784 | −0.7261882 | 0.19161038 |
| Gca | 69.9585093 | 1.501595 | 0.39647489 |
| Rab37 | 1776.46587 | 0.41011223 | 0.10852185 |
| Nr2f6 | 94.8683391 | 1.44085723 | 0.38146892 |
| Hk2 | 57.2674494 | −2.2330466 | 0.59157264 |
| G0s2 | 108.029131 | −1.9339849 | 0.51403044 |
| Gm37248 | 149.22326 | −2.2703852 | 0.60352541 |
| Acy1 | 266.061008 | −1.1046326 | 0.29437067 |
| Ube2g2 | 2822.61322 | 0.32166891 | 0.08580225 |
| Puf60 | 5782.11232 | 0.293298 | 0.07835565 |
| Dennd3 | 234.301776 | −1.5937497 | 0.42596795 |
| Sla2 | 1948.35338 | 0.34150636 | 0.09133507 |
| Rab32 | 114.705008 | 1.43812609 | 0.38524311 |
| Als2cl | 408.638035 | −1.3151762 | 0.35345036 |
| Ech1 | 2905.35271 | 0.39685877 | 0.10672085 |
| Gm35037 | 107.446913 | 1.06763526 | 0.2876618 |
| Pear1 | 1163.44353 | 0.67788406 | 0.18264084 |
| Pvr | 406.95226 | −0.9587068 | 0.2585974 |
| D630039A03Rik | 58.5310149 | 2.41265181 | 0.65228747 |
| Akr1c13 | 734.889468 | 0.64071704 | 0.1734438 |
| Gimap4 | 19739.7737 | 0.3734459 | 0.1011741 |
| Trim14 | 750.591047 | 0.63561677 | 0.17250337 |
| Cx3cr1 | 1993.16964 | 0.68434841 | 0.18587353 |
| Tmem229b | 1404.8993 | −0.4700454 | 0.12771075 |
| Agpat2 | 531.743956 | 0.5204873 | 0.14167997 |
| Add3 | 3490.76816 | −0.352779 | 0.09608435 |
| Gpr15 | 51.2775054 | 2.01964003 | 0.5511382 |
| Polr2e | 2706.22638 | 0.2953753 | 0.08066073 |
| Apol10b | 154.232348 | 1.49238543 | 0.40762381 |
| Sbfl | 2223.40728 | −0.5773083 | 0.15786242 |
| Ralb | 132.023867 | 1.06696754 | 0.2920349 |
| Ndfip1 | 5874.20357 | 0.39595281 | 0.10838806 |
| Notch1 | 632.523889 | −1.0649708 | 0.29173942 |
| Stk4 | 2897.4816 | −0.3966735 | 0.10871108 |
| Gpr183 | 2895.90698 | −0.3930702 | 0.1077673 |
| Glp1r | 920.83269 | 5.85858037 | 1.60840975 |
| Ctsc | 3626.26697 | 0.40005254 | 0.10984037 |
| Fmnl3 | 253.105348 | 1.43146932 | 0.39419304 |
| Nab1 | 2455.09738 | 0.4837122 | 0.13334998 |
| Pik3cd | 7347.59131 | −0.4603066 | 0.12692059 |
| Card6 | 522.244758 | −0.7169956 | 0.19780252 |
| Tpm4 | 3958.97064 | 0.41361391 | 0.11449689 |
| Clint1 | 3842.22475 | −0.3451369 | 0.09564083 |
| Mast3 | 1670.23687 | −0.5350514 | 0.14846165 |
| Tmem71 | 2646.3766 | −0.2828352 | 0.07853465 |
| Fmr1 | 562.524701 | −0.6273556 | 0.17423691 |
| Apc | 310.716265 | −0.8067719 | 0.22410597 |
| Actn1 | 201.52002 | −1.4511818 | 0.40326524 |
| Il10rb | 2022.78921 | 0.34078669 | 0.09483895 |
| Susd3 | 828.800205 | 0.51205397 | 0.14259869 |
| Suco | 686.365383 | −0.6209071 | 0.1729612 |
| Arhgef18 | 2534.12769 | −0.4408273 | 0.12283696 |
| Il17ra | 4261.17256 | −0.4707287 | 0.1312503 |
| Sypl | 458.450776 | 0.67441314 | 0.18812233 |
| Trpv2 | 2817.77735 | 0.43430976 | 0.12115942 |
| Smpdl3a | 3170.0109 | 0.38907526 | 0.10860835 |
| Etnk1 | 720.431551 | −0.7545918 | 0.21068814 |
| Ddc | 56.064097 | −2.1052854 | 0.58862428 |
| Map4k2 | 1451.77054 | −0.5073731 | 0.14192743 |
| Acp5 | 4297.20762 | 0.35118264 | 0.09845582 |
| Susd6 | 1013.7416 | −0.6739736 | 0.18907821 |
| Tafa3 | 62.4088185 | −1.4983569 | 0.4204719 |
| Gss | 351.057121 | −0.718525 | 0.201745 |
| Myc | 2087.95845 | 0.68583693 | 0.19265594 |
| Myl4 | 58.4335812 | −2.2554887 | 0.63368288 |
| Ifit3 | 666.686815 | 0.65635219 | 0.1846258 |
| Smyd1 | 218.347152 | −1.3538071 | 0.38074766 |
| Ugcg | 2042.67354 | −0.4815396 | 0.13553014 |
| Man1a2 | 803.906451 | −0.5390068 | 0.15179115 |
| Tent5a | 831.46917 | −0.5791548 | 0.1632334 |
| Slc25a46 | 684.346151 | −0.5250622 | 0.14805292 |
| Ccl3 | 3000.31304 | 1.06394422 | 0.30009296 |
| Ltbp4 | 178.970591 | 1.43252399 | 0.40609281 |
| Ubb | 16354.7255 | 0.36224479 | 0.10267276 |
| Pdia4 | 2655.43017 | 0.40251874 | 0.11424352 |
| Gm26740 | 1317.32143 | −0.6701888 | 0.19049256 |
| Tespa1 | 2159.5231 | −0.5103863 | 0.14527007 |
| Kbtbd3 | 208.657264 | 0.80666355 | 0.22964152 |
| Specc1 | 86.6303212 | −4.4305997 | 1.2621261 |
| Pea15a | 2157.75339 | 0.39247934 | 0.11187882 |
| Osgin1 | 188.248655 | 2.60900108 | 0.74386336 |
| Rasgrp1 | 2628.63989 | −0.3834738 | 0.10940085 |
| Vamp8 | 2255.32965 | 0.42881993 | 0.12238884 |
| Sccpdh | 90.4402462 | 2.51840982 | 0.71912876 |
| Jtb | 1637.31282 | 0.3479204 | 0.09938562 |
| Gpr55 | 103.250531 | −1.7408418 | 0.49749911 |
| Usp18 | 1711.64083 | 0.53403223 | 0.15264823 |
| Ttc17 | 759.769982 | −0.5916743 | 0.16919502 |
| Galnt6 | 1731.47849 | −0.5230067 | 0.14967697 |
| Zfp518a | 369.168636 | −0.8111599 | 0.23261749 |
| Tex2 | 137.918653 | −1.0823796 | 0.31046439 |
| Zfp106 | 588.595842 | −0.6589579 | 0.18926442 |
| Rnf166 | 4283.07388 | 0.31135113 | 0.08944777 |
| Klri2 | 89.9664696 | 4.03615781 | 1.16018469 |
| Trgc3 | 46.9458817 | 6.36721334 | 1.83128789 |
| Zfp445 | 685.223211 | −0.8639258 | 0.24844226 |
| Trak2 | 304.817868 | −1.0244054 | 0.29454331 |
| Epsti1 | 3667.00199 | −0.3647479 | 0.10500482 |
| Ar | 138.04856 | 1.4620485 | 0.42155011 |
| Optn | 43.4604904 | 2.85400865 | 0.82309623 |
| Ranbp1 | 891.894296 | 0.47610631 | 0.13744913 |
| Gm10522 | 185.615115 | −1.0902928 | 0.31501435 |
| Fam78a | 837.719011 | −0.6905291 | 0.19963627 |
| Zdhhc17 | 193.661415 | −0.9424603 | 0.27263023 |
| Snx4 | 1695.81586 | −0.4903584 | 0.14213993 |
| F730043M19Rik | 232.39801 | 0.99988304 | 0.28989785 |
| Ccr7 | 1912.42919 | −0.5497399 | 0.15970335 |
| Slc25a15 | 125.595632 | −1.2003816 | 0.34867832 |
| Trdc | 66.5452314 | 4.97293611 | 1.44625974 |
| Baiap3 | 5333.51897 | 0.57452181 | 0.16755362 |
| Klf7 | 245.348688 | −0.8895777 | 0.25963528 |
| Kcnc1 | 233.557579 | 1.27246656 | 0.37156684 |
| Tnfrsf13b | 169.170142 | −2.9138622 | 0.85081498 |
| Coro2a | 674.485928 | 0.8077556 | 0.23605118 |
| Zfp654 | 523.018858 | −0.6334213 | 0.18525077 |
| Timp2 | 234.928248 | 1.47309365 | 0.43103467 |
| Unc93b1 | 891.809785 | 0.57348204 | 0.16791307 |
| Il4ra | 1660.29764 | −0.3625443 | 0.10623303 |
| Spry2 | 18.1891568 | 3.13199381 | 0.91858653 |
| Ikzf1 | 2818.53141 | −0.4650118 | 0.13646601 |
| Frrs1 | 835.597365 | −0.4996341 | 0.14672129 |
| Fndc3a | 425.593595 | −0.8498876 | 0.24989178 |
| Hdac7 | 1218.87328 | −0.4317531 | 0.12723473 |
| Apbb1 | 260.552493 | 0.89693201 | 0.26489367 |
| Etv3 | 382.00064 | −0.8484966 | 0.25082841 |
| Enpp2 | 190.453442 | 1.68903493 | 0.49975988 |
| Eif4g3 | 848.38839 | −0.564736 | 0.16708856 |
| Snrnp200 | 1964.59456 | −0.6754842 | 0.2001823 |
| Sfr1 | 1648.5769 | 0.39079396 | 0.11599987 |
| Cdkn2c | 249.6315 | 0.94699518 | 0.281336 |
| Bptf | 928.736611 | −0.4558081 | 0.1355384 |
| Gnpat | 1393.63176 | −0.3552829 | 0.10572265 |
| Sidt2 | 2365.14535 | −0.2700502 | 0.0804306 |
| Ssh2 | 1648.56512 | −0.4665433 | 0.13895633 |
| Stk32c | 54.5996168 | 2.33297573 | 0.69500379 |
| Zmat1 | 243.247641 | 0.72184 | 0.2151122 |
| Gm15987 | 32.7752338 | 1.83691595 | 0.54786263 |
| 1700001O22Rik | 204.999676 | 0.88923745 | 0.26523105 |
| Nedd9 | 2618.63636 | −0.4089856 | 0.12205645 |
| Siah1a | 391.857368 | −0.6212938 | 0.18587571 |
| Acacb | 73.1072719 | 2.90381141 | 0.86909237 |
| Gbp3 | 871.918304 | 0.49049026 | 0.14716138 |
| Srsf9 | 1046.25539 | 0.469675 | 0.14104068 |
| Ifit2 | 341.512769 | −0.8068649 | 0.24251315 |
| Lrrfip2 | 444.98287 | 0.65735629 | 0.1977279 |
| Tmbim1 | 475.412229 | 0.6548954 | 0.19706508 |
| Ppcs | 619.695899 | 0.46850318 | 0.14094333 |
| BC147527 | 1264.54785 | 0.39936261 | 0.12016848 |
| Trav7-4 | 43.0665715 | −3.8268685 | 1.15219178 |
| Cerk | 424.102639 | −0.8740623 | 0.26324901 |
| Smg1 | 896.102779 | −0.4968427 | 0.14988831 |
| Dipk1b | 108.967074 | 1.37095912 | 0.41383754 |
| Ift140 | 356.502744 | −0.7769502 | 0.2346126 |
| 2410022M11Rik | 103.26872 | 1.14155037 | 0.34482714 |
| 2610507B11Rik | 1023.64074 | −0.5827173 | 0.17605919 |
| S100a4 | 664.059328 | 0.76883927 | 0.23238138 |
| Glo1 | 928.480019 | 0.45390451 | 0.13717976 |
| Tmem127 | 1108.03931 | −0.637858 | 0.19281849 |
| Cd47 | 10014.9726 | −0.3197369 | 0.09671364 |
| Zfp292 | 846.511219 | −0.6952634 | 0.2103212 |
| Mtg2 | 840.845456 | 0.46474132 | 0.14079544 |
| Nsmf | 413.047921 | 0.90274795 | 0.27361631 |
| Dnajc2 | 944.095517 | 0.45215142 | 0.13708544 |
| Dgka | 6549.47326 | −0.2953259 | 0.08971862 |
| Herc1 | 822.815156 | −0.6245425 | 0.19030413 |
| Rab3a | 135.920945 | 0.90937845 | 0.2772806 |
| Panx1 | 1648.77139 | 0.38321162 | 0.11683033 |
| Gm53055 | 54.77018 | 2.33939559 | 0.71372259 |
| Eif4ebp2 | 572.380843 | −0.5543223 | 0.16913576 |
| Slc22a15 | 256.503485 | 1.48598094 | 0.45356498 |
| Gpr155 | 297.154226 | 0.83711355 | 0.25564371 |
| Tmed7 | 1317.52849 | −0.3571071 | 0.10905224 |
| Fam3b | 42.0832 | 1.54245943 | 0.47133772 |
| Ryk | 215.172056 | 0.93708768 | 0.2863773 |
| Anp32b | 1567.31771 | 0.35054682 | 0.10710959 |
| Tm9sf3 | 4568.41814 | −0.2921732 | 0.08925988 |
| Dgkd | 842.403417 | −0.627784 | 0.19189918 |
| N4bp2 | 69.0524851 | −1.3960092 | 0.42706331 |
| Tbx6 | 28.1888911 | −2.5808255 | 0.78954986 |
| Ankrd44 | 2058.48758 | −0.5277382 | 0.161581 |
| Usp40 | 250.68124 | −1.0377529 | 0.31812712 |
| Gm527 | 52.8102598 | 1.54552014 | 0.47397627 |
| Zc3h6 | 120.636245 | −0.9853659 | 0.30247078 |
| Serpinb6a | 778.077299 | 0.57173709 | 0.17566264 |
| Insl6 | 673.495886 | 0.51041286 | 0.15691199 |
| Ulk1 | 254.345106 | −0.9792037 | 0.30098559 |
| Prdm9 | 96.0483189 | 1.10377167 | 0.33947304 |
| Map3k2 | 350.047677 | −0.6421505 | 0.19754992 |
| Ltk | 87.6530381 | 2.02600974 | 0.62385676 |
| Mast2 | 207.909772 | −1.0970983 | 0.3377855 |
| Gmfb | 1227.0662 | −0.3844913 | 0.11843144 |
| Kdm6a | 892.059004 | −0.7167228 | 0.22077821 |
| 4932438A13Rik | 1787.07695 | −0.6576032 | 0.20268919 |
| Zfpl1 | 341.632555 | 0.65543428 | 0.20221025 |
| Actn2 | 174.565912 | −1.6542855 | 0.51035899 |
| Ptk2b | 3274.91285 | −0.306624 | 0.09471115 |
| Polr3f | 334.865003 | −0.7646021 | 0.23620586 |
| Mroh2a | 129.508955 | −3.2196075 | 0.99482837 |
| Ipo11 | 361.431825 | −0.706656 | 0.21845155 |
| Dmtf1 | 879.943277 | −0.4776959 | 0.14781569 |
| Calcrl | 188.92291 | −1.1405154 | 0.352868 |
| Arid1b | 502.126716 | −0.6972687 | 0.21588184 |
| Kremen1 | 240.626522 | −1.1893013 | 0.36881528 |
| Sec16a | 507.783707 | −0.8782678 | 0.27307773 |
| Card19 | 730.240929 | 0.48144124 | 0.15046132 |
| Ubn1 | 901.414797 | −0.4337311 | 0.13558856 |
| Slc37a2 | 251.197084 | 1.32077871 | 0.41334521 |
| Nde1 | 1281.74686 | 0.40168565 | 0.12581848 |
| Man2a2 | 527.303949 | −0.8931666 | 0.28012864 |
| Ndufaf4 | 602.657547 | 0.53138372 | 0.16670555 |
| Sipa1 | 5951.18885 | 0.45745855 | 0.14365019 |
| Tbcel | 231.814807 | −0.913686 | 0.28724695 |
| Pacs1 | 1725.73445 | −0.4351263 | 0.13684876 |
| Kansl1 | 1117.16096 | −0.5414432 | 0.17027936 |
| Asap2 | 131.827206 | 1.17055023 | 0.36850037 |
| Fntb | 198.627581 | −0.8135363 | 0.25632702 |
| 1810037I17Rik | 1795.9979 | 0.37225355 | 0.11746593 |
| Rab27a | 710.496477 | 0.90957815 | 0.28708056 |
| Gbp11 | 33.6327182 | 3.4996053 | 1.10523893 |
| Plekhg2 | 493.234171 | −0.623072 | 0.19671752 |
| Serinc5 | 63.1694065 | −2.9565653 | 0.93367253 |
| Abi2 | 184.425917 | −1.1649413 | 0.36896711 |
| Zfp318 | 234.047241 | −0.8225313 | 0.26058223 |
| Larp4b | 821.77217 | −0.4649841 | 0.1473981 |
| Smpdl3b | 1573.24442 | 0.43206999 | 0.13702821 |
| Fcho1 | 2933.90378 | 0.32498908 | 0.1030813 |
| Rnf167 | 4144.86446 | −0.2844285 | 0.09022168 |
| Lcmt2 | 447.718558 | 0.53686306 | 0.17048393 |
| Hivep3 | 218.279167 | 0.92720183 | 0.29463194 |
| Tmem231 | 56.8765821 | −1.5919009 | 0.5059194 |
| Pkm | 7034.77165 | 0.26200748 | 0.08332288 |
| Kdm5a | 1896.6751 | −0.3981519 | 0.12676516 |
| Tec | 350.925913 | −0.6220989 | 0.19830887 |
| Plxna1 | 62.7994806 | 1.77328018 | 0.5655185 |
| Paqr4 | 138.078436 | 1.31013103 | 0.41798324 |
| Dusp2 | 2575.14259 | 0.42990069 | 0.13714478 |
| Hexa | 987.72307 | 0.36584546 | 0.11672133 |
| Kctd17 | 137.387502 | 0.92678007 | 0.29589955 |
| Siah2 | 294.816507 | −0.6851876 | 0.21903522 |
| Rbm33 | 375.209242 | −0.7199552 | 0.23045018 |
| Smad1 | 246.060426 | 1.0488656 | 0.33580689 |
| Adgrb2 | 55.5698308 | 1.62245775 | 0.51973524 |
| Clybl | 293.978481 | 0.60400478 | 0.193515 |
| Lcn4 | 532.554389 | −0.6983114 | 0.22378796 |
| Ip6k2 | 192.22755 | 0.9484622 | 0.30429715 |
| Zfp605 | 152.969812 | −1.0481367 | 0.3363094 |
| Lmf1 | 386.635007 | 0.56693253 | 0.18201817 |
| Cnp | 4730.41778 | −0.3154815 | 0.10133228 |
| Srsf7 | 4138.43761 | 0.34480454 | 0.11083631 |
| Thap12 | 736.653695 | −0.6653516 | 0.2139703 |
| Mrgbp | 568.762062 | 0.43961722 | 0.14153029 |
| Dnajb11 | 2232.104 | 0.31089241 | 0.10014624 |
| Sacs | 335.768835 | −1.2054054 | 0.38833958 |
| Elmo1 | 3738.67186 | −0.3127422 | 0.10078835 |
| Extl3 | 319.233394 | −0.7960363 | 0.25651173 |
| Ubxn4 | 1574.01476 | 0.33452324 | 0.10785644 |
| B4galt1 | 877.289269 | −0.473188 | 0.15254115 |
| Mrpl38 | 1037.02119 | 0.43467886 | 0.14023376 |
| Pts | 790.664018 | 0.35431486 | 0.11433103 |
| Irf2 | 1995.23572 | 0.34567194 | 0.11162404 |
| Gvin-ps7 | 3288.46417 | −0.4290556 | 0.13852457 |
| Ift172 | 257.431873 | −1.4669411 | 0.47370717 |
| Hnrnpl | 2864.20412 | −0.4076084 | 0.13164753 |
| Ppp1r12a | 2731.79786 | −0.3129199 | 0.10109704 |
| Serpinb1a | 190.83529 | 0.96787714 | 0.31316579 |
| Pum2 | 490.926141 | −0.5800564 | 0.18766863 |
| Gm17435 | 76.9693558 | −1.0488004 | 0.3393398 |
| Mob3a | 3240.54415 | 0.32904172 | 0.10660619 |
| Sin3a | 755.104916 | −0.4780675 | 0.15491025 |
| Madd | 1954.06751 | −0.5653493 | 0.18321167 |
| Gpaa1 | 1414.8037 | 0.37995911 | 0.12325712 |
| Zfp322a | 315.981011 | −0.6717993 | 0.217997 |
| Cst3 | 1954.87772 | 0.36462926 | 0.11849265 |
| Trabd | 1464.70957 | 0.3530863 | 0.11481409 |
| Ext1 | 102.688272 | −1.1772988 | 0.38313998 |
| Lax1 | 4039.04056 | 0.30856579 | 0.1004387 |
| Pik3r5 | 2743.78837 | −0.366545 | 0.11935156 |
| Cnot1 | 872.804992 | −0.5807245 | 0.18911292 |
| Atp6v1g1 | 961.45982 | 0.4429472 | 0.14439912 |
| Zbtb7b | 163.061912 | −1.4024873 | 0.45717093 |
| Arhgap1 | 2911.37423 | −0.2193032 | 0.07151722 |
| Rtn4rl1 | 373.901237 | −0.673442 | 0.22013395 |
| Ubac2 | 2617.08804 | 0.35431236 | 0.11590445 |
| Slamf7 | 2744.85162 | 0.33761999 | 0.11049633 |
| Zfp239 | 139.734289 | 0.99422303 | 0.32564101 |
| Ap1g1 | 591.97301 | −0.7101512 | 0.23263098 |
| Srp72 | 998.263982 | −0.5684478 | 0.18650143 |
| Tmem237 | 128.567641 | 0.78880293 | 0.25896609 |
| Utp11 | 756.344346 | 0.44656617 | 0.14674366 |
| Thada | 820.975496 | −0.4318506 | 0.1418737 |
| Insig1 | 101.607905 | −1.1566764 | 0.38006671 |
| Crmp1 | 381.921611 | 1.03690345 | 0.3411759 |
| Sf3b1 | 7255.32568 | −0.4357744 | 0.14338659 |
| Cxcr3 | 8678.27479 | 0.31155267 | 0.10254966 |
| Fnta | 1618.1021 | 0.29377684 | 0.09673728 |
| Cdk19 | 302.259433 | −0.8188256 | 0.26969955 |
| Zbtb1 | 1026.29126 | −0.4920936 | 0.16216399 |
| Mrpl41 | 616.470762 | 0.46201283 | 0.15232069 |
| Rexo1 | 1488.47963 | −0.4226427 | 0.13932568 |
| Tubgcp6 | 297.314845 | −0.894649 | 0.29491313 |
| Tmsb4x | 113097.642 | 0.25167966 | 0.08301 |
| Ptprk | 114.775536 | −1.7659371 | 0.58244503 |
| Septin11 | 1882.77357 | 0.37844257 | 0.12491022 |
| Anxa5 | 1969.45139 | 0.36289856 | 0.1197941 |
| Mbnl2 | 617.937691 | −0.5731136 | 0.18919199 |
| Casp4 | 329.71306 | 0.65253015 | 0.21584542 |
| Cnot6 | 957.983681 | −0.49282 | 0.16317633 |
| Trp53i13 | 564.475677 | 0.4356516 | 0.14429302 |
| Dhrs7 | 1593.95813 | 0.40621653 | 0.13456764 |
| St6galnac6 | 37.3590152 | −2.3321215 | 0.77310955 |
| Igkc | 2942.94265 | 0.93289699 | 0.30956478 |
| Mdm4 | 875.26955 | −0.473915 | 0.1574863 |
| Zfp120 | 513.36598 | 0.65892484 | 0.21906266 |
| Ddx43 | 136.508389 | 1.14980651 | 0.38257645 |
| Smpd5 | 961.203545 | 0.64540777 | 0.21470059 |
| Zmym3 | 235.209185 | −1.0168286 | 0.33830223 |
| Farp1 | 137.409684 | −1.130332 | 0.37594358 |
| NA | 56.0464411 | 1.48058763 | 0.49393721 |
| Mcl1 | 3743.1938 | −0.429088 | 0.14316688 |
| Crtc3 | 404.29289 | −0.5515238 | 0.18418922 |
| Asf1b | 295.437398 | 0.74089625 | 0.24751222 |
| Pcna | 1276.19623 | 0.30369126 | 0.10151816 |
| Frmd4b | 321.267978 | −0.8622775 | 0.28817299 |
| Fan1 | 68.4464531 | −1.5143382 | 0.50622747 |
| Trio | 90.8038943 | −1.8299223 | 0.61339617 |
| Stmn1 | 265.237443 | 0.70750994 | 0.23749515 |
| Dpy30 | 869.998314 | 0.35707887 | 0.11985907 |
| Ubxn7 | 288.246918 | −0.9578479 | 0.32153136 |
| Dnajc15 | 3935.336 | −0.3194137 | 0.10724169 |
| Stambpl1 | 634.585481 | −0.4869498 | 0.16351812 |
| Eno1 | 15500.4405 | 0.31969317 | 0.10737804 |
| Cipc | 971.924284 | −0.3940014 | 0.13236377 |
| Trgv7 | 76.9508539 | 6.05546193 | 2.0354031 |
| Dck | 470.549324 | −0.6383965 | 0.21458808 |
| Atpif1 | 369.966634 | 0.56253132 | 0.18932722 |
| Calm2 | 6951.48889 | 0.3570245 | 0.12018611 |
| Sp3 | 1372.77159 | −0.5023272 | 0.16919083 |
| Fut8 | 865.208069 | −0.3866956 | 0.13034162 |
| Tpst2 | 6283.78731 | 0.25951254 | 0.08754731 |
| Abca7 | 1315.03132 | −0.5504967 | 0.18570211 |
| Prelid2 | 129.47534 | 0.97707708 | 0.32982179 |
| Myd88 | 1212.37243 | −0.4341545 | 0.14657147 |
| Zfp182 | 327.3791 | −0.6869892 | 0.23191965 |
| Nr1d2 | 780.374621 | −0.6909332 | 0.23321268 |
| B4galt7 | 1332.10103 | 0.33018595 | 0.11154589 |
| Pdlim1 | 2063.24649 | 0.32036016 | 0.10823821 |
| Kmt5b | 1214.57941 | −0.5637293 | 0.19050973 |
| Bscl2 | 2190.64199 | 0.36941663 | 0.12502956 |
| Hspa5 | 9267.30123 | 0.32891078 | 0.11132899 |
| Trappc8 | 899.05077 | −0.6313971 | 0.21370368 |
| Gigyf2 | 564.960614 | −0.647534 | 0.21913534 |
| Sertad3 | 471.141023 | −0.5167662 | 0.17504477 |
| Bcl3 | 586.993322 | 0.86864484 | 0.29434322 |
| S1pr4 | 1840.72343 | −0.355502 | 0.1205409 |
| Themis | 3238.02562 | −0.3810099 | 0.1292104 |
| Naa15 | 854.330431 | −0.4222878 | 0.14319573 |
| Slc43a2 | 242.099351 | −0.8893846 | 0.30167506 |
| Tmem184c | 480.159038 | 0.5865219 | 0.19897868 |
| Ikbke | 1604.35687 | −0.3449678 | 0.1173951 |
| Tgfbr2 | 2817.88857 | −0.3750259 | 0.12767579 |
| Pak2 | 1366.76519 | −0.4000912 | 0.13636289 |
| Gm19589 | 163.792643 | 0.95735945 | 0.32638649 |
| Cul3 | 2727.7041 | −0.2994294 | 0.10220384 |

What is claimed is:

1. A method of treating cancer comprising administering to a patient in need thereof an effective amount of CD8 positive stem-like chronic memory cells.

2. The method of claim 1, wherein the CD8 positive stem-like chronic memory cells are PD-1 positive, CD62L positive and CD127 positive.

3. The method of claim 1, wherein the CD8 positive stem-like chronic memory cells are replicated ex vivo prior to administration.

4. The method of claim 1, wherein the CD8 positive stem-like chronic memory cells or replicated cells thereof are derived from the patient or derived from a person other than the patient.

5. The method of claim 4, wherein the CD8 positive stem-like chronic memory cells or replicated cells thereof are derived from a person other than the patient who recovered from a cancer therapy.

6. The method of claim 1, wherein the CD8 positive stem-like chronic memory cells comprise a recombinant vector encoding a chimeric antigen receptor.

7. The method of claim 1, wherein the CD8 positive stem-like chronic memory cells are administered in combination a checkpoint inhibitor.

8. The method of claim 1, wherein the checkpoint inhibitor is an anti-PD1 antibody or anti-PD-L1 antibody.

9. The method of claim 8, wherein the checkpoint inhibitor is an anti-PD1 antibody or anti-PD-L1 antibody is selected from pembrolizumab, nivolumab, cemiplimab, atezolizumab, dostarlimab, durvalumab, and avelumab.

10. A composition of CD8 positive stem-like chronic memory cells made by the process of purifying cells from a sample that are PD-1 positive and CD8 positive providing PD1 and CD8 positive cells; purifying cells from the PD1 and CD8 positive cells that are CD62L positive providing PD-1, CD8, CD62L, and CD127 positive cells.

11. A method of treating chronic viral infection comprising administering to a subject in need thereof an effective amount of CD8 positive stem-like chronic memory cells.

12. The method of claim 11, wherein the chronic viral infection is selected from HBV, HCV, and HIV.

13. The method of claim 11, wherein the composition of cells is administered in combination with another antiviral agent.

14. The method of claim 11, wherein the CD8 positive stem-like chronic memory cells are CD62L positive and CD127 positive.

15. The method of claim 11, wherein the CD8 positive stem-like chronic memory cells are replicated ex vivo prior to administration.

16. The method of claims 11, wherein the CD8 positive stem-like chronic memory cells or replicated cells thereof are derived from the patient or derived from a person other than the patient.

17. A method of isolating CD8 positive stem-like chronic resource cells comprising,
obtaining a sample from a subject,
purifying cells in the sample that are PD-1 positive and CD8 positive providing PD1 and CD8 positive cells;
purifying cells from the PD-1 and CD8 positive cells providing cells that express TCF1, are CD44 positive, and have no or low expression of Tim3, CD39 negative, or combination of these markers or other markers as disclosed herein, providing isolated CD8 positive stem-like chronic resource cells.

18-30. (canceled)

31. The method of claim 1, wherein the CD8 positive stem-like chronic memory cells are PD-1 positive, CD62L positive, CD127 positive, and are cells that express TCF1, and CD44 positive, and CD39 negative.

32. The method of claim 10, wherein the CD8 positive stem-like chronic memory cells are PD-1 positive, CD62L positive, CD127 positive, and are cells that express TCF1, are CD44 positive, and CD39 negative.

33. The method of claim 11, wherein the CD8 positive stem-like chronic memory cells are PD-1 positive, CD62L positive, CD127 positive, and are cells that express TCF1, are CD44 positive, and CD39 negative.
